(19) United States
(12) Patent Application Publication
(10) Pub. No.: US 2026/0185018 A1
(43) Pub. Date: Jul. 2, 2026

(54) IMPROVED WASHING PERFORMANCE THROUGH THE USE OF A PROTEASE FUSED WITH A SPECIAL ADHESION PROMOTER PEPTIDE

(71) Applicant: Henkel AG & Co. KGaA, Duesseldorf (DE)

(72) Inventors: Shohana Islam, Alsbach-Hähnlein (DE); Christian Degering, Erkrath (DE); Susanne Wieland, Zons/Dormagen (DE); Anna Ebel, Bergisch Gladbach (DE); Iva Anic, Düsseldorf (DE); Irmgard Schmidt, Solingen (DE); Roland Breves, Mettmann (DE); Christian Kastner, Düsseldorf (DE)

(21) Appl. No.: 19/128,006

(22) PCT Filed: Nov. 13, 2023
(86) PCT No.: PCT/EP2023/081557

§ 371 Date: May 7, 2025

(30) Foreign Application Priority Data
Nov. 30, 2022 (DE) 10 2022 131 732.3

(51) Int. Cl. C11D 3/386; C12N 9/54; C12N 9/96

(52) U.S. Cl. CPC C11D 3/38681; C12N 9/54; C12N 9/96; C12Y 304/21062

(57) ABSTRACT

The invention is in the field of enzyme technology. The invention relates to proteases which are covalently bonded with a heterologous peptide sequence, as a result of which the proteases have a better cleaning performance. The invention further relates to the uses of these protease conjugates and methods in which they are deployed, as well as agents, in particular washing and cleaning agents, containing said proteases.

The invention is in the field of enzyme technology. The invention relates to proteases which are covalently bonded with a heterologous peptide sequence, as a result of which the proteases have a better cleaning performance. The invention further relates to the uses of these protease conjugates and methods in which they are deployed, as well as agents, in particular washing and cleaning agents, containing said proteases.

Proteases are among the technically most important enzymes. For washing and cleaning agents, they are the longest established enzymes and are contained in virtually all modem, high-performance washing and cleaning agents. They cause the degradation of protein-containing stains on the articles to be cleaned. In turn, proteases of the subtilisin type (subtilases, subtilopeptidases, EC 3.4.21.62) are particularly important, which proteases are serine proteases due to the catalytically active amino acids. They act as unspecific endopeptidases and hydrolyze any acid amide bonds within peptides or proteins. Their optimum pH is usually in the distinctly alkaline range. The article “Subtilases: Subtilisin-like Proteases” by R. Siezen, pages 75-95 in “Subtilisin enzymes,” published by R. Bott and C. Betzel, New York, 1996, gives an overview of this family, for example. Subtilases are naturally formed by microorganisms. In particular, the subtilisins formed and secreted by the Bacillus species are the most significant group of subtilases.

Examples of subtilisin-type proteases that are preferably used in washing and cleaning agents are the subtilisins BPN′ from Bacillus amyloliquefaciens and Carlsberg from Bacillus licheniformis, protease PB92, subtilisins 147 and 309, the protease from Bacillus lentus, in particular Bacillus lentus DSM 5483, subtilisin DY and the enzymes thermitase, proteinase K, and proteases TW3 and TW7, which are to be classified as subtilases, but no longer as subtilisins in the narrower sense, as well as variants of said proteases that have an amino acid sequence which is modified with respect to the starting protease. Proteases are altered in a targeted or random manner by methods known from the prior art and are thus optimized for use in washing and cleaning agents, for example. These include point mutagenesis, deletion or insertion mutagenesis or fusion with other proteins or protein parts. Thus, appropriately optimized variants are known for most proteases known from the prior art. Subtilisin Carlsberg is available in a developed form under the trade name Alcalase® from Novozymes. Subtilisins 147 and 309 are marketed by Novozymes under the trade names Esperase® and Savinase®, respectively. The protease variants marketed under the name BLAP® are derived from the protease from Bacillus lentus DSM 5483. Other proteases that can be used are, for example, the enzymes available under the trade names Durazym®, Relase®, Everlase®, Nafizym®, Natalase®, Kannase® and Ovozyme® from Novozymes, the enzymes available under the trade names Purafect®, Purafect® OxP, Purafect® Prime, Excellase® and Properase® from Danisco/Genencor, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease P® from Amano Pharmaceuticals Ltd., and the enzyme available under the name Proteinase K-16 from Kao Corp. The proteases from Bacillus gibsonii and Bacillus pumilus, which are disclosed in international patent applications WO 2008/086916 and WO 2007/131656, as well as EP 2016175 are also particularly preferably used. Further proteases that can be used advantageously are disclosed in patent applications WO 91/02792, WO 2008/007319, WO 93/18140, WO 01/44452, GB 1243784, WO 96/34946, WO 2002/029024, and WO 2003/057246. Further proteases that can be used are those which are naturally present in the microorganisms Stenotrophomonas maltophilia, in particular Stenotrophomonas maltophilia K279a, Bacillus intermedius and Bacillus sphaericus.

In general, only selected proteases are suitable for use in liquid surfactant-containing preparations. Many proteases do not exhibit sufficient catalytic performance in such preparations. For the use of proteases in cleaning agents, therefore, a high catalytic activity under conditions as they are during a wash cycle and a high storage stability is particularly desirable.

Consequently, protease and surfactant-containing liquid formulations from the prior art are disadvantageous in that the proteases contained, under standard washing conditions (e.g. in a temperature range of from 20° C. to 40° C.), do not have satisfactory proteolytic activity or are not storage-stable and the formulations therefore do not exhibit optimal cleaning performance on protease-sensitive stains.

Surprisingly, it has now been found that a protease as defined herein, in particular a protease from Bacillus pumilus, or a sufficiently similar protease (in terms of sequence identity), in combination with a covalently bonded, heterologous adhesion promoter peptide, is improved in terms of cleaning performance compared to the protease itself and is therefore particularly suitable for use in washing or cleaning agents.

Surprisingly, the inventors of the present invention have found that the performance of proteases can be significantly increased if they are fused with special adhesion-promoting peptides. This effect has been demonstrated herein using an example protease, but it can also be transferred to other proteases. The performance of the proteases is increased by fusion with the peptides described herein, which act as adhesion promoters, such that the concentration used can be significantly reduced without loss of performance.

Therefore, in a first aspect, the present invention is directed to a protease conjugate consisting of
A) a protease, preferably a protease of the subtilisin type, in particular from Bacillus pumilus, wherein the protease has proteolytic activity;
B) at least one heterologous peptide covalently linked to the protease; and optionally
C) at least one linker, preferably at least one peptide linker, preferably one peptide linker;
wherein the heterologous peptide is a peptide comprising or consisting of an amino acid sequence of 4 to 50 amino acids, preferably 10 to 24 amino acids, wherein
(a) the amino acid sequence in N- to C-terminal orientation has the following sequence

(C)ₘX₁X₂X₃(X₄)ₙX₅(C)ₒ,
where X₁ is a positively charged amino acid, preferably R or K, more preferably R, X₂ and X₃ are uncharged amino acids, preferably selected from A, L, S, I, M and Q, more preferably from A, S, I and L, in particular A and L, each X₄ is, independently of one another, any amino acid, preferably with the exception of P, more preferably with the exception of P and G; X₅ is any positively charged or uncharged amino acid, preferably R or an uncharged amino acid, more preferably Q, A or L, in particular A or L, m and o are 0 or 1, where m+o=0 or 1; and n is an integer from 0 to 46, preferably from 6 to 20;
or
(b) the amino acid sequence has at least 80%, preferably at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 19-20 or 21-25.

In a further aspect, the present invention is directed to a nucleic acid that codes for a protease conjugate according to the present invention.

In a further aspect, the present invention also relates to a non-human host cell that contains a nucleic acid according to the present invention or a protease conjugate according to the present invention.

In a yet further aspect, the present invention relates to a method for preparing a protease conjugate according to the invention, comprising:
a) cultivating a host cell according to the present invention; and
b) isolating the protease conjugate from the culture medium or from the host cell.

In a further aspect, the present invention is also directed to an agent, in particular a washing or cleaning agent, characterized in that it contains at least one protease conjugate according to the present invention.

In yet another aspect, the present invention is directed to methods for cleaning textiles or hard surfaces, characterized in that an agent according to the present invention is used in at least one method step.

Finally, the present invention in a final aspect is also directed to the use of a protease conjugate according to the present invention in a washing or cleaning agent for removing peptide- or protein-containing stains.

Advantageous embodiments and further developments are the subject of the dependent claims and the accompanying description.

Unless indicated otherwise, all percentages are indicated in terms of weight percent (wt. %).

Numeric ranges specified in the format “from x to y” include the specified values. If several preferred numerical ranges are specified in this format, it is readily understood that any ranges resulting from the combination of the various endpoints are also included.

“At least one,” as used herein, means 1 or more, e.g., 1, 2, 3, 4, 5, 6, 7, 8, 9 or more. In relation to a constituent or a compound, unless stated otherwise, this expression does not refer to the absolute number of molecules, but rather to the number of different types of molecules that fall under the relevant definition of the constituent or compound. “At least one protease” thus means that at least one type of protease is contained, and not that at least one protease molecule is contained.

“Approximately,” “about,” or “roughly,” as used herein in reference to a numerical value, refers to the corresponding numerical value ±10%, preferably ±5%.

“Substantially free of” means that the composition or the agent contains less than 2 wt. %, preferably less than 1 wt. %, more preferably less than 0.5 wt. %, and particularly preferably less than 0.1 wt. %, of the corresponding substance, relative to the total weight of the composition/agent.

“Liquid,” as used herein, includes liquids and gels as well as pasty compositions. It is preferred that the liquid compositions be flowable and pourable at room temperature, but it is also possible for them to have a limit of liquidity. A substance, e.g., a composition or an agent, is solid according to the definition of the invention if it is in a solid state of aggregation at 25° C. and 1,013 mbar. A substance, e.g., a composition or an agent, is liquid according to the definition of the invention if it is in a liquid state of aggregation at 25° C. and 1,013 mbar. Liquid also includes gel form.

“Heterologous,” as used herein with reference to the peptide sequence, refers to the fact that the peptide sequence, which has an adhesion-promoting effect, does not naturally occur in combination with the protease. The conjugates described herein are thus non-natural hybrids of a protease and a peptide not associated with the protease.

In the context of this invention, “adhesion” is understood to mean an interaction between the peptide and a surface, thereby allowing the peptide to adhere to the surface. Thus, “adhesion-promoting” refers to the ability to interact with different surfaces, i.e., textile surfaces, and/or to adhere to a particular surface under appropriate conditions, i.e., usually non-denaturing conditions, wherein a binding affinity is greater than that of a reference sequence that does not have adhesion-promoting properties.

The term “variant,” as used herein, refers to variants of an enzyme or a protein/peptide which continue to have the functionality of the starting molecule but differ from the starting sequence by one or more sequence deviations, for example 1, 2 or 3 or more sequence deviations, for example a substitution, deletion or insertion. The sequence identity of such variants may be in the range of 80% based on the total length of the starting peptide, and may be at least 81%, 82%, 83%, 84%, 85%, 86%, 87%, 88%, 89%, 90%, 91%, 92%, 93%, 94%, 95%, 96%, 97%, 98%, 99%, or 99.5%.

When reference is made herein to various bonded or individual amino acid sequences, unless stated otherwise, these sequences are always expressed in the N- to C-terminal orientation. Furthermore, unless stated otherwise, the individual amino acids or amino acid sequences are bonded to one another by means of peptide bonds. Accordingly, the hyphen in the designation protease-linker-peptide, for example, means that these three corresponding sequences are fused together via peptide bonds.

The identity of nucleic acid or amino acid sequences is determined by a sequence comparison. This sequence comparison is based on the BLAST algorithm, which is established and commonly used in the prior art (cf., for example, Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402) and in principle occurs in that similar sequences of nucleotides or amino acids in the nucleic acid or amino acid sequences are assigned to one another. A tabular assignment of the relevant positions is referred to as an alignment. A further algorithm available in the prior art is the FASTA algorithm. Sequence comparisons (alignments), in particular multiple sequence comparisons, are created using computer programs. Frequently used are for example the Clustal series (cf., for example, Chenna et al. (2003): Multiple sequence alignment with the Clustal series of programs. Nucleic Acid Research 31, 3497-3500), T-Coffee (cf., for example, Notredame et al. (2000): T-Coffee: A novel method for multiple sequence alignments. J. Mol. Biol. 302, 205-217) or programs based on these programs or algorithms are frequently used. Also possible are sequence comparisons (alignments) using the computer program Vector NTI® Suite 10.3 (Invitrogen Corporation, 1600 Faraday Avenue, Carlsbad, California, USA) with the specified standard parameters, the AlignX module of which for the sequence comparisons is based on ClustalW, or Clone Manager 10 (use of the scoring matrix BLOSUM 62 for sequence alignment at amino acid level). Unless stated otherwise, the sequence identity indicated herein is determined using the BLAST algorithm.

Such a comparison also allows a conclusion to be drawn about the similarity of the compared sequences to one another. It is usually indicated in percent identity, i.e., the proportion of identical nucleotides or amino acid functional groups in said sequences or in an alignment of corresponding positions. In the case of amino acid sequences, the broader concept of homology takes conserved amino acid exchanges into account, i.e., amino acids having similar chemical activity, as these usually perform similar chemical activities within the peptide/protein. Therefore, the similarity of the compared sequences can also be indicated as percent homology or percent similarity. Identity and/or homology information can be provided regarding whole peptides, polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Such small regions, however, often perform essential functions for the overall activity of the peptide/protein. It may therefore be expedient to relate sequence matches only to individual, optionally small, regions. Unless otherwise stated, however, identity or homology information in the present application relates to the total length of the respective nucleic acid or amino acid sequence indicated.

The peptide or protein concentration can be determined using known methods, for example the BCA method (bicinchoninic acid; 2,2′-bichinolyl-4,4′-dicarboxylic acid) or the Biuret method (A. G. Gornall C. S. Bardawill and M. M. David, J. Biol. Chem., 177 (1948), pp. 751-766). A person skilled in the art in the field of peptide and protein technology is aware of a variety of suitable methods for determining peptide or protein concentration that can be used within the scope of this invention.

The peptides may have amino acid changes, in particular amino acid substitutions, insertions or deletions. Such peptides are, for example, further developed by targeted genetic alteration, i.e., by mutagenesis methods, and optimized for specific purposes or with regard to specific properties (for example with regard to their stability, binding etc.). For example, targeted mutations such as substitutions, insertions or deletions can be introduced into the known molecules in order to alter certain properties, for example. For this purpose, in particular the surface charges and/or the isoelectric point of the molecules and thus their interactions with a surface can be altered. For example, the net charge of the peptides can be altered in order to thereby influence the substrate binding. Alternatively or additionally, one or more corresponding mutations can, for example, increase the stability or adsorption of the peptide. Advantageous properties of individual mutations, e.g., individual substitutions, can complement one another.

The term “conservative amino acid substitution” means the exchange (substitution) of one amino acid functional group for another amino acid functional group, wherein this exchange does not lead to a change in the polarity or charge at the position of the exchanged amino acid, for example the exchange of one non-polar amino acid functional group for another non-polar amino acid functional group. Conservative amino acid substitutions within the scope of the invention comprise, for example: G=A=S, I=V=L=M, D=E, N=Q, K=R, Y=F, S=T, G=A=I=V=L=M=Y=F=W=P=S=T. However, it may be preferred for such exchanges not to have glycine or tyrosine as the target amino acid or, for example, also not to have an amino acid which has a low alpha-helix-forming potential.

A protease conjugate according to the invention consists of
A) a protease, protease, preferably a protease of the subtilisin type, in particular from Bacillus pumilus, wherein the protease has proteolytic activity;
B) at least one heterologous peptide covalently linked to the protease; and optionally
C) at least one linker, preferably at least one peptide linker, preferably one peptide linker;
wherein the heterologous peptide is a peptide comprising or consisting of an amino acid sequence of 4 to 50 amino acids, preferably 10 to 24 amino acids, wherein
(a) the amino acid sequence in N- to C-terminal orientation has the following sequence

(C)ₘX₁X₂X₃(X₄)ₙX₅(C)ₒ,
where X₁ is a positively charged amino acid, preferably R or K, more preferably R, X₂ and X₃ are uncharged amino acids, preferably selected from A, L, S, I, M and Q, more preferably from A, S, I and L, in particular A and L, each X₄ is, independently of one another, any amino acid, preferably with the exception of P, more preferably with the exception of P and G; X₅ is any positively charged or uncharged amino acid, preferably R or an uncharged amino acid, more preferably Q, A or L, in particular A or L, m and o are 0 or 1, where m+o=0 or 1; and n is an integer from 0 to 46, preferably from 6 to 20;
or
(b) the amino acid sequence has at least 80%, preferably at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified SEQ ID NOs: 19-20 or 21-25.

### A) Protease

Proteases which can be used as a component of the protease conjugate according to the invention include all known and as yet unknown proteases, in particular those which are known in the prior art.

The proteases that are suitable in the context of the present invention are proteases which have enzymatic activity, i.e., they are capable of hydrolyzing peptides and proteins, in particular in a washing or cleaning agent. A suitable protease is therefore an enzyme that catalyzes the hydrolysis of amide/peptide bonds in protein/peptide substrates and is thereby capable of cleaving proteins or peptides. Furthermore, a suitable protease is preferably a mature protease, i.e., the catalytically active molecule without a signal peptide/signal peptides and/or a propeptide/propeptides. Unless otherwise stated, the sequences indicated also refer to mature (processed) enzymes in each case.

Examples of proteases are the subtilisins BPN′ from Bacillus amyloliquefaciens and Carlsberg from Bacillus licheniformis, protease PB92, subtilisins 147 and 309, the protease from Bacillus lentus, subtilisin DY, and the enzymes thermitase, proteinase K and proteases TW3 and TW7, which in the narrower sense are associated with the subtilases but no longer with the subtilisins. Subtilisin Carlsberg is available in a developed form under the trade name Alcalase® from Novozymes. Subtilisins 147 and 309 are marketed by Novozymes under the trade names Esperase® and Savinase®, respectively. The protease variants are derived from the protease from Bacillus lentus DSM 5483. Other proteases that are suitable are, for example, the enzymes available under the trade names Durazym®, Relase®, Everlase®, Nafizym®, Natalase®, Kannase®, Progress Uno 101L® and Ovozyme® from Novozymes, the enzymes available under the trade names Purafect®, Purafect® OxP, Purafect® Prime, Excellase® and Properase®, Preferenz P100® and Preferenz P300® from Danisco/DuPont, the enzyme available under the trade name Lavergy pro 104 LS® from BASF, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease P® from Amano Pharmaceuticals Ltd., and the enzyme available under the name Proteinase K-16 from Kao Corp. The proteases from Bacillus gibsonii and Bacillus pumilus, which are disclosed in WO 2008/086916, WO 2007/131656, WO 2017/215925, WO 2021/175696 and WO 2021/175697, are particularly preferably used. Further proteases which can be used advantageously are disclosed in e.g., WO 91/02792, WO 2008/007319, WO 93/18140, WO 01/44452, GB 1243784 A, WO 96/34946, WO 02/029024, and WO 03/057246. Further proteases that can be used are those which are naturally present in the microorganisms Stenotrophomonas maltophilia, in particular Stenotrophomonas maltophilia K279a, Bacillus intermedius and Bacillus sphaericus.

Preferred proteases comprise
a) a protease that has proteolytic activity and comprises an amino acid sequence which is at least 70% identical, and increasingly preferably at least 71%, 72%, 73%, 74%, 75%, 76%, 77%, 78%, 79%, 80%, 81%, 82%, 83%, 84%, 85%, 86%, 87%, 88%, 89%, 90.5%, 91%, 91.5%, 92%, 92.5%, 93%, 93.5%, 94%, 94.5%, 95.5%, 96%, 96.5%, 97%, 97.5%, 98%, 98.5% or 98.8% identical to the amino acid sequence given in SEQ ID NO:1 over its entire length and, respectively based on the numbering according to SEQ ID NO:2 has, (i) at the position corresponding to position 101, the amino acid substitution R101E, and (ii), at at least one of the positions corresponding to positions 3, 4, 45, 55, 58, 59, 61, 87, 97, 98, 106, 117, 120, 124, 129, 136, 137, 143, 156, 161, 163, 171, 172, 185, 199, 205, 209, 222, 238, 244, 261 and 262, at least one amino acid substitution selected from the group consisting of S3T, V4I, R45E, R45D, R45Q, P55N, T58W, T58Y, T58L, Q59D, Q59M, Q59N, Q59T, G61D, G61R, S87E, G97S, A98D, A98E, A98R, S106A, S106W, N117E, H120V, H120D, H120K, H120N, S124M, P129D, E136Q, Q137H, S143W, S156D, S161T, S163A, S163G, Y171L, A172S, N185Q, V199M, V205I, Y209W, M222Q, N238H, V244T, N261T and L262N, L262Q, L262D, L262E; wherein the amino acid substitution combination of group (ii) is preferably selected from the group consisting of N238E-L262E, S156D-L262E, S3T-V41-V2051, S3T-V41-A228V, G195E-V199M, H120D-S163G-N261D, N76D-A228V-N261D, S3T-N76D-S156D-Y209W, Q137H-S141H-R145H-N238E-L262E, Q137H-S141H-R145H-S156D-L262E, N76D-Q137H-S141H-R145H-A228V-N261D, N76D-Q137H-S141H-R145H-S163G-N238E, H120D-Q137H-S141H-R145H-S163G-N261D, S3T-N76D-Q137H-S141H-R145H-S156D-Y209W;
b) a protease that has proteolytic activity and comprises an amino acid sequence which is at least 70% identical, and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, and 98% identical to the amino acid sequence given in SEQ ID NO:3 over its entire length and, respectively based on the numbering according to SEQ ID NO:2 has, (i) at the positions corresponding to positions 9, 130, 133, 144, 217, 252 and 271, at least one amino acid substitution selected from the group consisting of P9T, N130D, N130V, T133A, N144K, Y217M, N252T and Q271E, and (ii), at at least one of the positions corresponding to positions 6, 61, 62, 63, 89, 99, 101, 131, 156, 166, 170, 187, 188, 189, 211 and 224, at least one amino acid substitution selected from the group consisting of Y6F, Y6W, F61G, Q62N, S63Q, S89A, S89G, N99H, D101S, D101E, D101A, G131H, G131Y, G131F, S156R, G166A, G166M, G166L, G1661, K170R, K170G, N187D, N188G, S189T, S189L, S1891, S189R, S211N, S211Q, S224A, S224G, wherein the protease preferably has an amino acid substitution combination selected from the group consisting of P9T-N130D-T133A-N144K-G166M-S189T-Y217M-N252T-Q271E, P9T-N130D-T133A-N144K-G166M-S189T-Y217M-S224A-N252T-Q271E, P9T-N130D-T133A-N144K-G166M-S211N-Y217M-N252T-Q271E, P9T-S89A-N130D-G131H-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-N187D-Y217M-S224A-N252T-Q271E, P9T-S89A-N130D-T133A-N144K-S189R-Y217M-S224A-N252T-Q271E, P9T-S89A-N130D-T133A-N144K-N187D-S189R-Y217M-S224A-N252T-Q271E, P9T-S89A-D101S-N130D-T133A-N144K-S189T-Y217M-S224A-N252T-Q271E, P9T-S89A-D101E-N130D-T133A-N144K-S189T-Y217M-S224A-N252T-Q271E, P9T-S89A-D101A-N130D-T133A-N144K-S189T-Y217M-S224A-N252T-Q271E, P9T-S89A-N99H-N130D-T133A-N144K-K170R-S189T-Y217M-S224A-N252T-Q271E, P9T-S89A-N130D-T133A-N144K-S156R-S189T-Y217M-S224A-N252T-Q271E, P9T-S63Q-S89A-N130D-T133A-N144K-G166A-S189T-Y217M-S224A-N252T-Q271E, P9T-F61G-Q62-N-S89A-N130D-T133A-N144K-N188G-S189T-Y217M-S224A-N252T-Q271E, Y6W-P9T-N130D-T133A-N144K-S189T-Y217M-S224A-N252T-Q271E, Y6W-P9T-S89A-N130D-T133A-N144K-S189T-Y217M-S224A-N252T-Q271E, Y6W-P9T-F61G-Q62N-S89A-N130D-T133A-N144K-S189T-Y217M-S224A-N252T-Q271E, Y6W-P9T-S89A-N130D-T133A-N144K-N188G-S189T-Y217M-S224A-N252T-Q271E;
c) a protease which has proteolytic activity and comprises an amino acid sequence which is at least 70% identical and increasingly preferably at least 71%, 72%, 73%, 74%, 75%, 76%, 77%, 78%, 79%, 80%, 81%, 82%, 83%, 84%, 85%, 86%, 87%, 88%, 89%, 90.5%, 91%, 91.5%, 92%, 92.5%, 93%, 93.5%, 94%, 94.5%, 95.5%, 96%, 96.5%, 97%, 97.5%, 98%, 98.5%, or 98.8% identical to the amino acid sequence given in SEQ ID NO:1 over its entire length and, respectively based on the numbering according to SEQ ID NO:1 has (i), at the positions corresponding to positions 3, 4, 99 and 199, the amino acid substitutions S3T, V41, R99E and V199I, and (ii), at at least one of the positions corresponding to positions 74, 136, 143, 154, 160, 161, 163, 171, 181, 183, 185, 200, 203, 209, 212 or 256, at least one amino acid substitution selected from the group consisting of N74D, N74E, N74Q, A136Q, R143L, R143W, R143Y, S154D, S154Q, S160G, Y161T, A163G, V171L, A181D, F183R, Q185R, Q200A, Q200L, Q200S, Q200T, Y203K, Y203V, Y203W, A209K, A209W, N212S, N212T and L256D, L256E and L256Q, wherein the protease preferably has an amino acid substitution combination selected from the group consisting of S3T-V4I-R99E-V199I-Q200L-Y203W, S3T-V4I-R99E-V199I-N212S, S3T-V4I-R99E-V199I-N74D, S3T-V4I-R99E-V199I-S154D-L256E, S3T-V4I-R99E-V199I-Q200L-Y203W-S154D-L256E, S3T-V41-R99E-V199I-N74D-Q200L-Y203W, S3T-V4I-R99E-V199I-N74D-S154D-Q200L-Y203W-L256E, S3T-V4I-R99E-V199I-N74D-N212S, S3T-V4I-R99E-V199I-N74D-S154D-Y203W-L256E, S3T-V4I-R99E-V199I-N74D-Y203W, S3T-V4I-R99E-V199I-N74D-S154D-Q200L-L256E, S3T-V4I-R99E-V199I-N74D-Q200L, S3T-V4I-R99E-V199I-S154D-Q200L-Y203W, S3T-V41-R99E-V199I-Q200L-Y203W-L256E, S3T-V4I-R99E-V199I-A136Q-R143W-Y161T-Q200L, S3T-V4I-R99E-V199I-N74D-R143Y-A209W-N212S-L256E, S3T-V41-R99E-V199I-N74D-S154D-Y203W-L256E; S3T-V4I-R99E-V199I-Q200L-Y203W-A209K-S154D-L256E, S3T-V4I-R99E-V199I-S154D-S160G-Q185R-Q200L-Y203W-L256E, S3T-V41-R99E-V199I-S154D-A181D-F183R-Q200L-Y203W-L256E, and S3T-V4I-R99E-V199I-A136Q-S154D-V171L-Q200L.

Preferred in the context of the present invention are proteases of the subtilisin type, in particular those from Bacillus pumilus, as disclosed in DE 102020105721 A1, DE 102020205400 A1, DE 102016204814 A1, DE 102016208463 A1, DE 102017215628 A1 and DE 102017215629 A1.

Particularly preferably, the protease is selected from proteases of group b).

Most preferably, the protease is a protease which has proteolytic activity and comprises an amino acid sequence that is at least 70% identical and increasingly preferably at least 71%, 72%, 73%, 74%, 75%, 76%, 77%, 78%, 79%, 80%, 81%, 82%, 83%, 84%, 85%, 86%, 87%, 88%, 89%, 90.5%, 91%, 91.5%, 92%, 92.5%, 93%, 93.5%, 94%, 94.5%, 95.5%, 96%, 96.5%, 97%, 97.5%, and 98% identical to the amino acid sequence given in SEQ ID NO:3 over its entire length and, respectively based on the numbering according to SEQ ID NO: 2, (i), at the positions corresponding to positions 9, 130, 133, 144, 217, 252 and 271, has at least one amino acid substitution selected from the group consisting of P9T, N130D, N130V, T133A, N144K, Y217M, N252T and Q271E, and (ii), at at least one of the positions corresponding to positions 6, 61, 62, 63, 89, 99, 101, 131, 156, 166, 170, 187, 188, 189, 211 and 224, at least one amino acid substitution selected from the group consisting of Y6F, Y6W, F61G, Q62N, S63Q, S89A, S89G, N99H, D101S, D101E, D101A, G131H, G131Y, G131F, S156R, G166A, G166M, G166L, G1661, K170R, K170G, N187D, N188G, S189T, S189L, S1891, S189R, S211N, S211Q, S224A, S224G, wherein the protease preferably has the following amino acid substitution combination: P9T-S89A-N130D-T133A-N144K-S189T-Y217M-S224A-N252T-Q271E.

### B) Peptide

The heterologous peptide bonded according to the invention to the protease as described and defined above, which peptide acts as an adhesion mediator for the protease, is a peptide comprising or consisting of an amino acid sequence of 4 to 50 amino acids, preferably of at least 8, 9, 10, 11 or 12 amino acids in length. Preferred lengths are up to 40, up to 35, up to 30, or up to 25, or up to 24 amino acids. For example, the peptide may have a length of 11, 12, 13, 14, 15, 16, 17, 18, 19, 20, 21, 22, 23, or 24 amino acids, in particular 12 to 18 amino acids.

The peptides have
(a) in N- to C-terminal orientation the following amino acid sequence

(C)ₘX₁X₂X₃(X₄)ₙX₅(C)ₒ,
where X₁ is a positively charged amino acid, preferably R or K, more preferably R, X₂ and X₃ are uncharged amino acids, preferably selected from A, L, M, S, I and Q, more preferably from A, S, I and L, in particular from A and L, each X₄ is, independently of one another, any amino acid, preferably with the exception of P, more preferably with the exception of P and G; X₅ is any positively charged or uncharged amino acid, preferably R or an uncharged amino acid, more preferably Q, A or L, in particular A or L, m and o are 0 or 1, where m+o=0 or 1; and n is an integer from 0 to 46, preferably from 6 to 20, e.g., 9, 10, 11, 12 or 13.

Alternatively or additionally, the peptides comprise or consist of an amino acid sequence having at least 80%, preferably at least 81%, at least 82%, at least 83%, at least 84%, or at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to one of the amino acid sequences specified in SEQ ID NOs: 16-17 and/or 18-22.

By a “peptide” in the context of the present invention is meant a polymer composed of amino acids, preferably the 20 proteinogenic L-amino acids, preferably of linear structure, which has up to 100 amino acids linked together by peptide bonds. The peptides of the invention have an amino acid sequence of 4 to 50 amino acids. In the context of the present invention, amino acids are indicated in single-letter code, where, for example, C is cysteine, R is arginine, A is alanine, and L is leucine. In particular, “C” in the above sequence (C)ₘX₁X₂X₃(X₄)ₙX₅(C)ₒ represents a cysteine residue. It is further understood that unless otherwise indicated, the amino acids in an amino acid sequence disclosed herein are linked via peptide bonds and the sequence is set forth in N- to C-terminal orientation unless otherwise indicated.

In various embodiments, the peptides may have been chemically synthesized and/or recombinantly produced by means of protein design. Nowadays, short peptides can be easily synthesized, for example via solid-phase synthesis. In contrast, longer peptides and polypeptides are often also produced recombinantly in host organisms.

In the context of the present invention, the term “N-terminus” or “N-terminal” typically describes the end of the amino acid chain of the peptide that has a free amino group.

In the context of the present invention, the term “C-terminus” or “C-terminal” typically describes the end of the amino acid chain of the peptide that has a free carboxyl group.

In the context of the present invention, the term “in N- to C-terminal orientation” refers to an amino acid sequence in which the sequence of amino acids are described starting from the N-terminus towards the C-terminus.

Typical acidic or negatively charged amino acids (depending on pH) are D and E.

Positively charged or basic amino acids (depending on pH) typically include R, K, and H.

Amino acids such as G, A, C, I, L, M, F, V, P, S, T, W, Y, N and Q are typically uncharged, i.e., neutral amino acids.

When reference is made herein to “any” amino acid, this usually means one of the 20 naturally occurring proteinogenic amino acids, i.e., one of glycine (G), alanine (A), valine (V), leucine (L), isoleucine (I), phenylalanine (F), serine (S), threonine (T), proline (P), methionine (M), cysteine (C), histidine (H), lysine (K), arginine (R), glutamine (Q), asparagine (N), aspartic acid (D), glutamic acid (E), tyrosine (Y) and tryptophan (W). The amino acids are typically L-amino acids unless otherwise specified. In alternative embodiments, the peptide may also consist of D-amino acids, although it may be preferred that, within the peptides described herein, D- and L-amino acids are not simultaneously present. In various embodiments, any such amino acid encompasses all of the aforementioned amino acids with the exception of proline, or in some embodiments, also with the exception of proline and glycine. In certain embodiments, these two amino acids are not preferred because they have helix-breaking properties and may therefore adversely affect the secondary structure of the peptides.

In preferred embodiments, the peptide has a total charge of from −2 to +12, preferably from 0 to +8, more preferably from 0 to +4, in particular from 0 to +2. The total charge of the peptide is based on the number of positively and negatively charged amino acids in the peptide, in particular arginine (R), lysine (K), histidine (H), aspartic acid (D), and glutamic acid (E), and is the sum of the negative and positive charges, wherein one positive and one negative charge cancel each other out. Thus, a peptide with 2 arginine residues and 1 glutamic acid residue would have a total charge of +1. The total charge of the peptide is preferably −2 to +12, more preferably 0 to +8, even more preferably 0 to +4, in particular 0 to +2.

In preferred embodiments, the peptide has
(i) a total charge of from −2 to +12, preferably from 0 to +8, more preferably from 0 to +4, in particular from 0 to +2; and/or
(ii) at the N-terminus comprising the first 3-4 amino acids, a net positive charge, and/or
(iii) at the C-terminus comprising the last 3-6 amino acids, a net negative or neutral charge, preferably at least one negatively charged amino acid, in particular E; and/or
(iv) no P and even more preferably also no G and in particular no Y.

All aforementioned features, in particular (i)-(iv), may be realized individually or in any combination.

The feature that the peptide at the “N-terminus comprising the first 3-4 amino acids has a net positive charge” means that the N-terminal 3-4 amino acids comprise more positively charged amino acids than negatively charged amino acids. In various embodiments, this feature is fulfilled, for example, when the N-terminal 3-4 amino acids have 1 or 2 positively charged amino acids, i.e., H, K or R, preferably K or R, more preferably R, and no negatively charged amino acids, such as E or D. If the N-terminus contains a negatively charged amino acid, the number of positively charged amino acids must be at least 2 in order for the net charge to remain positive.

For the feature that the peptide at the “C-terminus comprising the last 3-6 amino acids has a net negative or neutral charge” means that the number of charged amino acids must be 0 or the number of negatively amino acids, i.e., D and E, must be greater than the number of positively charged ones. An example of such a C-terminal sequence would be, for example, EAL or the double sequence of this motif.

In various preferred embodiments, the sequence is X₁X₂X₃ RAL, RSI, or RLA, preferably RAL or RLA, in particular RAL. The N-terminal sequence RAL or RLA not only advantageously has a net positive charge, it also comprises amino acids with a particularly high alpha-helix-forming potential, as will be explained further below. In some embodiments, the arginine residue may also be replaced by lysine, but the N-terminal arginine residue is particularly preferred.

Furthermore, it is preferred that
(i) the sequence (X₄)ₙX₅ comprises at least one sequence X₆X₇X₈, where X₆ is a charged or uncharged amino acid, preferably R, K, E, L, A or Q, more preferably R, K, E or Q, and X₇ and X₈ are, independently from one another, negatively charged or uncharged amino acids with the exception of P and G, preferably A, L, E, R, Q or M, for example A, L, E, Q or M, even more preferably A, E, Q or L, even more preferably A, E or L, in particular A or L; and/or (ii) (X₄)ₙ comprises at least one aromatic amino acid, preferably W or F.

If the sequence X₆X₇X₈ comprises an X₆ which is R or K, the sequence X₆X₇X₈ is preferably not at the C-terminus and preferably not within the 6 C-terminal amino acids. In such cases, the sequence (X₄)ₙX₅ may comprise one or more further sequences X₆X₇X₈, which are C-terminal to the sequence, which comprise X₆ a positively charged amino acid, wherein these further sequences then preferably do not have a positively charged amino acid as X₆.

It is preferred that one of the sequences X₆X₇X₈, which are close to, i.e., within the 6 C-terminal amino acids, or at the C-terminus, have as X₆ a negatively charged amino acid, for example E.

If, in some embodiments, the peptide contains an aromatic amino acid selected from W and F, a positively or negatively charged amino acid is adjacent thereto, in particular C-terminally, and in particular no other aromatic amino acid is adjacent to the aromatic amino acid.

The aromatic amino acids phenylalanine (F) and tryptophan (W) are preferably used as helix formers and/or for pi-stacking in the peptide sequence according to the invention. In various embodiments the aromatic amino acid tyrosine (Y) is not used in the peptide sequence because it has helix-breaking properties. In various embodiments, the peptide is therefore free of Y residues.

In the context of the present invention, “Pi-stacking” refers to the non-covalent interaction between aromatic ring systems.

Preferably
(i) (X₄)ₙ comprises at least one sequence X₆X₇X₈, where X₆X₇X₈ is RAL or RLA, preferably RAL, and where said sequence is preferably localized in the N-terminal amino acids of positions 4-7, or at least 6-7 amino acids away from the C-terminus; and/or
(ii) (X₄)ₙX₅ comprises at least one sequence X₆X₇X₈, where X₆X₇X₈ is EAL, LEA or ELA, preferably EAL, and where said sequence is preferably not localized in the N-terminal amino acids of positions 1-6; and/or
(iii) (X₄)ₙX₅ comprises at least one sequence X₆X₇X₈, where X₆X₇X₈ is EQA, QAL, LQA or QLA, preferably EQA, QAL or QLA, in particular QAL.

In other possible embodiments, (X₄)ₙX₅ comprises at least one sequence X₆X₇X₈, where X₆X₇X₈ is QLA or EQA, where said sequence is preferably not localized in the N-terminal amino acids of positions 1-6 or 1-11.

It is further possible that (X₄)ₙX₅ comprises at least one sequence X₆X₇X₈X₉ where X₆X₇X₈X₉ is AQLA or SEQA, where said sequence is preferably not localized in the N-terminal amino acids of positions 1-6 or 1-11.

In preferred embodiments, the peptide comprises the sequence X₁X₂X₃, where X₁X₂X₃ is RAL, and (X₄)ₙX₅ comprises at least one of QAL and EAL, preferably both. In such embodiments, the peptide has the sequence

(C)ₘRAL(X₁₀)_{q}QAL(X₁₁)ᵣEAL(X₁₂)ₛ(C)ₒ,
or
(C)ₘRAL(X₁₀)_{q}EAL(X₁₁)ᵣQAL(X₁₂)ₛ(C)ₒ,

where X₁₀ and X₁₁ are, independently from one another, any amino acid, preferably with the exception of P, more preferably with the exception of P and G; X₁₂ is any uncharged amino acid, preferably Q, A or L, in particular A or L;q and r are 0 or integers from 1-10, preferably 0, 1, 2 or 3; and s is 0 or 1, where q+r+s=0-21, preferably 0-15 or 0-9 or 1-15 or 1-9 or 1-6 or 0-6 or 0-3 or 1-3.

Furthermore, it is preferred that the peptide additionally comprises at least one further (second) sequence RAL. Said sequence may, in various embodiments, directly C-terminally follow the first RAL sequence or be separated from it by 1-3 amino acids, for example 7 by 1 or 3 three amino acids.

In preferred embodiments, the peptide contains two sequences RAL and at least one sequence each of EAL and AL. Preferred sequences are:

RALRAL(X₁₀)_{q}QAL(X₁₁)ᵣEAL(X₁₂)ₛ,
RALRAL(X₁₀)_{q}EAL(X₁₁)ᵣQAL(X₁₂)ₛ
RAL(X₁₀)_{q}RALQAL(X₁₁)ᵣEAL(X₁₂)ₛ,
RAL(X₁₀)_{q}RALEAL(X₁₁)ᵣQAL(X₁₂)ₛ
RAL(X₁₀)_{q}QALRAL(X₁₁)ᵣEAL(X₁₂)ₛ,
RAL(X₁₀)_{q}EALRAL(X₁₁)ᵣQAL(X₁₂)ₛ,

where X₁₀ and X₁₁ are, independently of one another, any amino acid, preferably with the exception of P, more preferably with the exception of P and G, e.g., W or F or further subjects QAL or EAL; X₁₂ is any uncharged amino acid, preferably Q, A or L, in particular A or L; q and r are 0 or integers from 1-6, preferably 0, 1, 2, or 3; and s is 0 or 1, preferably 0, where q+r=0-9, preferably 0-6 or 0-3 or 1-9 or 1-6 or 1-3.

Furthermore, it is preferred in various embodiments that the peptide contains at least one W or F, preferably exactly one W or F.

Preferably, the peptide comprises amino acids having a high alpha-helix-forming potential, with said amino acids being selected from E, A, L, M, Q, K, R, F, I, H, W and D, more preferably E, A, L, M, Q, K, R, F, I and H; even more preferably E, A, L, M, Q, K, R and F.

In preferred embodiments, the peptide consists of at least 60%, preferably at least 65%, more preferably at least 70%, in particular at least 75% or at least 80% or at least 85% or at least 90% or at least 95% of amino acids having a high alpha-helix-forming potential, with said amino acids being preferably selected from E, A, L, M, Q, K, R, F, I, H, W and D, more preferably E, A, L, M, Q, K, R, F, I and H; even more preferably E, A, L, M, Q, K, R and F.

Particularly preferably, the peptide forms a helical secondary structure, in particular an α-helical structure, preferably with an α-helical content of at least 80%, preferably at least 85%, more preferably at least 90%, even more preferably at least 95%, in particular higher than 95%. The use of the AL or LA motif in the amino acid sequence of the peptide can contribute to the stability of the helix structure because these amino acids have a high a-helix potential.

In preferred embodiments, the peptide has an amino acid sequence according to one of SEQ ID NOs: 1-15 and/or 16-17, in particular 1-15, or variants thereof, which has at least 80%, preferably at least 81%, at least 82%, at least 83%, at least 84% or at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% sequence identity to the specified sequence, wherein preferably the RAL motif, and more preferably also the EAL and/or QAL motif, if present, are invariant. If the RAL, EAL and QAL motifs are present in the peptide, they are preferably invariable in all the aforementioned variants.

In various embodiments, the peptide may have a high proportion of hydrophobic amino acids selected from A, L, F, W, V, M, I, and P, in particular A, L, F, W, V, M, and I.

Preferably, the peptide has an amino acid sequence which has a length of 10 to 24 amino acids, for example 11, 12, 13, 14, 15, 16, 17, 18, 19, 20, 21, 22, 23, or 24 amino acids, in particular 12 to 19 amino acids, for example 12-18 amino acids.

In some embodiments, the peptide has the amino acid cysteine (C) at the C-terminus. In other embodiments, the peptide has the amino acid cysteine at the N-terminus. This amino acid can enable coupling to other molecules, structures or substrates via the free sulfhydryl group. This amino acid therefore serves as a linkage point but is typically not involved in the desired adhesive effect.

Preferably, the peptide comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: RALQALRALQALEAL (SEQ ID NO:4), RALRALRALEALEAL (SEQ ID NO:5), RALRALRALQALQAL (SEQ ID NO:6, RALRALRALQALEAL (SEQ ID NO:7), RALRALQALEALEAL (SEQ ID NO:8), RALFEALQALFRALEAL (SEQ ID NO:9), RALRALEALQALEA (SEQ ID NO:10), RALFEALFRALEALR (SEQ ID NO:11), RALFEALFRALEAL (SEQ ID NO:12), RALEALFRALEAL (SEQ ID NO:13), RALRALFEALEAL (SEQ ID NO:14), RALEALFRALQALEAL (SEQ ID NO:15), RALEALWRALQALEAL (SEQ ID NO:16), RALEALWRALEAL (SEQ ID NO:17), RALARALARALAQALA (SEQ ID NO:18), RSIVTFSLRQNAQLA (SEQ ID NO:19) or RSIVTFSLRQNSEQA (SEQ ID NO:20), preferably 4-18.

In various other embodiments, the peptide comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: RSIVTFSLRQNAQLA (SEQ ID NO: 19), RSIVTFSLRQNSEQA (SEQ ID NO: 20), GLHTSATNLYLH (SEQ ID NO: 21), QHSIRLLTIKKP (SEQ ID NO: 22), QQSIRIMTIKHP (SEQ ID NO: 23), WRHPRLRCGNLL (SEQ ID NO:24) or QKSRNRMTRTHP (SEQ ID NO: 25), preferably 19 or 20.

In various other embodiments, the peptide comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: GLHTSATNLYLH (SEQ ID NO:21), QHSIRLLTIKKP (SEQ ID NO:22), QQSIRIMTIKHP (SEQ ID NO:23), WRHPRLRCGNLL (SEQ ID NO:24) or QKSRNRMTRTHP (SEQ ID NO:25).

In various embodiments, the peptide may further comprise or consist of one of the following amino acid sequences, or a variant thereof: SRARLFVVTYHK (SEQ ID NO: 26), HMISTMNAASRR (SEQ ID NO: 27), RSIVTFSLRQNR (SEQ ID NO: 28), RNTIRIRTIKHP (SEQ ID NO: 29), or RHSSTLRYRPLP (SEQ ID NO: 30), wherein a variant has an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the amino acid sequences.

Variants of the peptides, the amino acid sequence of which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the amino acid sequences which correspond to the sequences in SEQ ID NOs: 4-30, preferably 4-25, more preferably 4-18, 19-20 or 21-25, more preferably 4-18 or 19-20, in particular 4-18, preferably differ in a maximum of 3 positions, more preferably in a maximum of 2 positions, in particular in a maximum of 1 position from one of the amino acid sequences, which correspond to the sequences in SEQ ID NOs: 4-30, preferably 4-25, more preferably 4-18, 19-20, or 21-25, more preferably 4-18 or 19-20, in particular 4-18. For example, if 1 position is different, either one of the amino acids of the respective sequence can be replaced or else the sequences match, but an amino acid within the amino acid chain or N- or C-terminal has been added or omitted. For example, a cysteine (C) could have been attached to the C- or N-terminal.

Thus, such peptides are also suitable which are characterized in that they are obtainable from a peptide as described above as starting molecule, for example from a molecule having one of the amino acid sequences according to SEQ ID NOs: 4-30, preferably 4-25, more preferably 4-18, 19-20 and/or 21-25, even more preferably 4-18 or 19-20, in particular 4-18, on which, for example, one or more amino acid substitutions, including, inter alia, single or multiple conservative amino acid substitutions, have been carried out, wherein the resulting peptide has at least 80% sequence identity to one of the amino acid sequences according to SEQ ID NOs: 4-30, preferably 4-25, more preferably 4-18, 19-20 or 21-25, even more preferably 4-18 or 19-20, in particular 4-18.

In preferred embodiments, the peptide may also be modified. Preferred modifications may include, for example, coupling the peptide to certain other molecules or chemical groups, e.g., organic (macro)molecules, including peptide molecules. If the peptide is coupled with at least one further (macro)molecule, it may also be referred to as a peptide derivative. The peptide is then derivatized.

In some embodiments, the mentioned peptides which may, for example for coupling purposes, N- and/or C-terminally have the amino acid cysteine, are coupled (functionally modified) with biotin, preferably at a suitable amino acid of the chain and/or N- and/or C-terminally. However, in the context of the present invention, such a modification is only possible at the N- or C-terminal terminus which is not covalently bonded to the protease, optionally by means of a linker C).

In various embodiments, the peptide may
(i) comprise or consist of any of the following amino acid sequences: RALQALRALQALEAL-C (SEQ ID NO: 31), RALRALRALEALEAL-C (SEQ ID NO: 32), RALRALRALQALQAL-C (SEQ ID NO: 33), RALRALRALQALEAL-C (SEQ ID NO: 34), RALRALQALEALEAL-C (SEQ ID NO: 35), RALFEALQALFRALEAL-C (SEQ ID NO: 36), RALRALEALQALEA-C (SEQ ID NO: 37), RALFEALFRALEALR-C (SEQ ID NO: 38), RALFEALFRALEAL-C (SEQ ID NO: 39), RALEALFRALEAL-C (SEQ ID NO: 40), RALRALFEALEAL-C (SEQ ID NO: 41), RALEALFRALQALEAL-C (SEQ ID NO: 42), RALEALWRALQALEAL-C (SEQ ID NO: 43), RALEALWRALEAL-C (SEQ ID NO: 44), RALARALARALAQALA-C (SEQ ID NO: 45), RSIVTFSLRQNAQLA-C (SEQ ID NO: 46), RSIVTFSLRQNSEQA-C (SEQ ID NO: 47), in particular one of SEQ ID NOs: 31-39, 42-47, in particular 31-35, 37, 45-47; or
(ii) comprise or consist of an amino acid sequence which has at least 80%, preferably at least 81%, 82%, 83%, 84% or 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the sequences specified in SEQ ID NOs: 31-47 (31, 32, 33, 34, 35, 36, 37, 38, 39, 40, 41, 42, 43, 44, 45, 46 or 47), preferably with one of SEQ ID NOs: 31-39, 42-47, in particular 31-35, 37, 45-47.

Furthermore, it is possible for the peptide to
(i) comprise or consist of one of the amino acid sequences: GLHTSATNLYLH-C (SEQ ID NO: 48), QHSIRLLTIKKP-C (SEQ ID NO: 49), QQSIRIMTIKHP-C (SEQ ID NO: 50), WRHPRLRCGNLL-C (SEQ ID NO: 51), or QKSRNRMTRTHP-C (SEQ ID NO: 52); or
(ii) comprises or consists of an amino acid sequence which has least 80%, preferably at least 81%, 82%, 83%, 84% or 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the sequences specified in SEQ ID NOs: 48-52 (48, 49, 50, 51 or 52).

In various embodiments, the peptide may further comprise or consist of an amino acid sequence according to SEQ ID NOs: 53-57 (SRARLFVVTYHK-C (SEQ ID NO: 53), HMISTMNAASRR-C (SEQ ID NO: 54), RSIVTFSLRQNR-C (SEQ ID NO: 55), RNTIRIRTIKHP-C- (SEQ ID NO: 56), RHSSTLRYRPLP-C (SEQ ID NO: 57)) or a variant thereof, which has at least 80%, preferably at least 81%, 82%, 83%, 84% or 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity therewith.

In the aforementioned embodiments in which the said peptides have the amino acid cysteine at the N- or C-terminus, the protease is covalently linked to the peptide at the other terminus of the peptide according to the invention. In other words, in the case of a cysteine bonded N-terminally to the peptide, the protease is correspondingly bonded to the C-terminus of the peptide, or in the case of a cysteine bonded C-terminally to the peptide, the protease is correspondingly bonded to the N-terminus of the peptide.

In various embodiments of the invention, the heterologous peptide sequence which functions as an adhesion agent for the protease is directly covalently linked to the protease, i.e., the first and/or last amino acid of the protease is linked to a terminal amino acid of the peptide sequence by means of a peptide bond. Alternatively, the binding can also be achieved by means of a linker, in particular a peptide linker. Such binding by means of a peptide linker is preferred. Suitable linkers are known in the prior art and can be static/rigid or flexible. This property is determined by the secondary structure of the linker; for example, rigid linkers can have an alpha helix as a secondary structure. In various embodiments, the peptide linker sequence is flexible and does not have a secondary structure or only has short secondary structural elements. Linkers suitable in the context of the present invention are described below.

### C) Linker

In the context of the present invention, suitable linkers, which are preferably peptide linkers, can be divided into flexible linkers and stiff/rigid linkers. In principle, linkers of this type are basically known from the prior art. If the protease conjugate according to the invention comprises at least one linker, preferably one linker (dear inventors, please check), then the linker provides the covalent bond of the heterologous peptide to the protease and the corresponding protease conjugate has the following structure in N- to C-terminal orientation:
(i) peptide linker protease; or
(ii) (ii) protease linker peptide.

If the linker is a peptide linker, as preferred, then the first or last amino acid of the protease is linked via a peptide bond to a terminal amino acid of the linker sequence and the first or last amino acid of the heterologous peptide is linked to the other terminal amino acid of the linker sequence.

Typically, peptide linkers of this kind have a length of from 1 to 200 amino acids, for example 1 to 100 amino acids, preferably from 2 to 30 amino acids, more preferably from 5 to 25 amino acids.

In various embodiments, the linker is selected from the group consisting of (I) (GGGGS)ₙ with n=1, 2, 3, or 4; (II) (G)₆; (G)₈; (III) (EAAAK)ₙ with n=1, 2, or 3; (IV) A(EAAAK)₄ALEA(EAAAK)₄A; (V) PAPAP; (VI) AEAAAKEAAAKA; and (VII) (AP)ₙ with n=10-34.

In the context of the present invention, functional homologs of the aforementioned linker sequences are also suitable.

“Functional homolog,” as used in this context, relates to sequences that are at least 70% identical, preferably 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4%, or 99.6% identical to the indicated reference sequence and have the functionality thereof, i.e., can function as a bond between protease and heterologous peptide without impairing the protease conjugates corresponding to the advantageous properties as described and disclosed herein.

Preferred combination of protease, linker and heterologous peptide are, in N- to C-terminal orientation:
(a) peptide linker (I) protease;
(b) peptide linker (II) protease;
(c) peptide linker (III) protease;
(d) peptide linker (IV) protease;
(e) peptide linker (V) protease;
(f) peptide linker (VI) protease;
(g) peptide linker (VII) protease;
(h) protease linker (I) peptide;
(i) protease linker (II) peptide;
(j) protease linker (III) peptide;
(k) protease linker (IV) peptide;
(l) protease linker (V) peptide;
(m) protease linker (VI) peptide;
(n) protease linker (VII) peptide,
wherein the protease preferably has one of the following amino acid substitution variants, in each case based on the numbering according to SEQ ID NO:1:
P9T, N130D, T133A, N144K, G166M, S189T, Y217M, N252T, Q271E;
P9T, N130D, T133A, N144K, G166M, S189T, Y217M, S224A, N252T, Q271E;
P9T, S89A, N130D, G131H, T133A, N144K, S189T, Y217M, S224A, N252T, Q271E;
Y6W, P9T, N130D, T133A, N144K, S189T, Y217M, S224A, N252T, Q271E;
P9T, N130D, T133A, N144K, G166M, S211N, Y217M, N252T, Q271E;
P9T, S89A, N130D, T133A, N144K, S189T, Y217M, S224A, N252T, Q271E;
P9T, S89A, N130D, T133A, N144K, N187D, Y217M, S224A, N252T, Q271E;
P9T, S89A, N130D, T133A, N144K, S189R, Y217M, S224A, N252T, Q271E;
P9T, S89A, N130D, T133A, N144K, N187D, S189R, Y217M, S224A, N252T, Q271E;
preferably: P9T, S89A, N130D, T133A, N144K, S189T, Y217M, S224A, N252T, Q271E,
wherein the peptide preferably comprises or consists of one of the following amino acid sequences, or is a variant thereof: RALQALRALQALEAL (SEQ ID NO:4), RALRALRALEALEAL (SEQ ID NO:5), RALRALRALQALQAL (SEQ ID NO:6), RALRALRALQALEAL (SEQ ID NO:7), RALRALQALEALEAL (SEQ ID NO:8), RALFEALQALFRALEAL (SEQ ID NO:9), RALRALEALQALEA (SEQ ID NO:10), RALFEALFRALEALR (SEQ ID NO:11), RALFEALFRALEAL (SEQ ID NO:12), RALEALFRALEAL (SEQ ID NO:13), RALRALFEALEAL (SEQ ID NO:14), RALEALFRALQALEAL (SEQ ID NO:15), RALEALWRALQALEAL (SEQ ID NO:16), RALEALWRALEAL (SEQ ID NO:17), RALARALARALAQALA (SEQ ID NO:18), RSIVTFSLRQNAQLA (SEQ ID NO:19) or RSIVTFSLRQNSEQA (SEQ ID NO:20), GLHTSATNLYLH (SEQ ID NO:21), QHSIRLLTIKKP (SEQ ID NO:22), QQSIRIMTIKHP (SEQ ID NO:23), WRHPRLRCGNLL (SEQ ID NO:24) or QKSRNRMTRTHP (SEQ ID NO:25), SRARLFVVTYHK (SEQ ID NO:26), HMISTMNAASRR (SEQ ID NO:27), RSIVTFSLRQNR (SEQ ID NO:28), RNTIRIRTIKHP (SEQ ID NO:29) or RHSSTLRYRPLP (SEQ ID NO:30), wherein the peptide preferably consists of one of the amino acid sequences SEQ ID NOs: 4-30.

In various embodiments, the above-mentioned combinations (a)-(n) are preferred, wherein the protease in each case has the amino acid substitution variant P9T, S89A, N130D, T133A, N144K, S189T, Y217M, S224A, N252T, Q271E, based on the numbering according to SEQ ID NO:1, and wherein the peptide in each case consists of one of the amino acid sequences SEQ ID NOs: 4-30.

A further subject matter of the present invention is a nucleic acid that codes for a protease conjugate according to the invention, and a vector containing such a nucleic acid, in particular a cloning vector or an expression vector.

These can be DNA or RNA molecules. They can be present as a single strand, as a single strand complementary to said single strand or as a double strand. In particular in the case of DNA molecules, the sequences of the two complementary strands must be taken into account in all three possible reading frames. Furthermore, it must be taken into account that different codons, i.e., base triplets, can code for the same amino acids such that a certain amino acid sequence can be coded by a plurality of different nucleic acids. Due to this degeneracy of the genetic code, all of the nucleic acid sequences which can code any of the proteases described above are included in this subject matter of the invention. A person skilled in the art is able to determine these nucleic acid sequences beyond a doubt because, despite the degeneracy of the genetic code, defined amino acids can be assigned to individual codons. Therefore, a person skilled in the art proceeding from said amino acid sequence can easily determine nucleic acids coding for said amino acid sequence. Furthermore, in the case of nucleic acids according to the invention, one or more codons can be replaced by synonymous codons. This aspect relates in particular to the heterologous expression of the enzymes according to the invention. Thus, each organism, for example a host cell of a production strain, has a certain codon usage. “Codon usage” is understood to mean the translation of the genetic code into amino acids by the relevant organism. Bottlenecks can occur in protein biosynthesis if the codons on the nucleic acid in the organism are faced with a comparatively small number of loaded tRNA molecules. Although coding for the same amino acid, this results in a codon being translated less efficiently in the organism than a synonymous codon coding for the same amino acid. Due to the presence of a higher number of tRNA molecules for the synonymous codon, this can be translated more efficiently in the organism.

It is possible for a person skilled in the art to use methods which are currently generally known, e.g. chemical synthesis or polymerase chain reaction (PCR), in conjunction with molecular biological and/or protein-chemical standard methods, to produce the corresponding nucleic acids and even complete genes on the basis of known DNA and/or amino acid sequences. Such methods are known, e.g., from Sambrook, J., Fritsch, E. F. and Maniatis, T. 2001. Molecular cloning: a laboratory manual, 3. Edition Cold Spring Laboratory Press.

For the purposes of the present invention, “vectors” are understood to mean elements consisting of nucleic acids that contain a nucleic acid according to the invention as the characteristic nucleic acid region. They are able to establish these as a stable genetic element in a species or cell line over several generations or cell divisions. Vectors are, in particular when used in bacteria, special plasmids, i.e., circular genetic elements. In the context of the present invention, a nucleic acid according to the invention is cloned into a vector. The vectors include, for example, those originating from bacterial plasmids, viruses or bacteriophages, or predominantly synthetic vectors or plasmids having elements of a wide variety of origins. With the other genetic elements present in each case, vectors are able to establish themselves as stable units in the particular host cells over several generations. They can be present extrachromosomally as separate units or can be integrated into a chromosome or chromosomal DNA.

Expression vectors comprise nucleic acid sequences that allow them to replicate in the host cells containing them, preferably microorganisms, particularly preferably bacteria, and to express a contained nucleic acid there. The expression is influenced in particular by the promoter or promoters that regulate transcription. In principle, the expression can take place by the natural promoter originally located before the nucleic acid to be expressed, but also by a promoter of the host cell provided on the expression vector or also by a modified or completely different promoter of another organism or another host cell. In the present case, at least one promoter is provided for the expression of a nucleic acid according to the invention and used for the expression thereof. Expression vectors can also be regulatable, for example by changing the cultivation conditions or when a certain cell density of the host cells containing them is reached or by adding certain substances, in particular activators of gene expression. An example of such a substance is the galactose derivative isopropyl P-D-thiogalactopyranoside (IPTG), which is used as an activator of the bacterial lactose operon (lac operon). In contrast to expression vectors, the nucleic acid contained is not expressed in cloning vectors.

The invention further relates to a non-human host cell containing a nucleic acid according to the invention or a vector according to the invention, or containing a protease conjugate according to the invention, in particular one that secretes the protease conjugate into the medium surrounding the host cell. Preferably, a nucleic acid according to the invention or a vector according to the invention is transformed into a microorganism that then represents a host cell according to the invention. Alternatively, individual components, i.e., nucleic acid parts or fragments of a nucleic acid according to the invention, can also be introduced into a host cell in such a way that the resulting host cell contains a nucleic acid according to the invention or a vector according to the invention. This procedure is particularly suitable when the host cell already contains one or more constituents of a nucleic acid according to the invention or a vector according to the invention and the further constituents are then supplemented accordingly. Methods for transforming cells are established in the prior art and are well known to a person skilled in the art. In principle all cells, i.e., prokaryotic or eukaryotic cells, are suitable as host cells. Host cells which can be managed in a genetically advantageous manner, for example with regard to transformation with the nucleic acid or the vector and its stable establishment, are preferred, for example single-cell fungi or bacteria. Furthermore, preferred host cells are distinguished by good microbiological and biotechnological manageability. This relates, for example, to easy cultivation, high growth rates, low requirements for fermentation media and good production and secretion rates for foreign proteins. Preferred host cells according to the invention secrete the (transgenically) expressed protein into the medium surrounding the host cells. Furthermore, the proteases can be modified by the cells producing them after their production, for example by linking sugar molecules, formylations, aminations, etc. Such post-translational modifications can functionally influence the protease conjugate.

Further preferred embodiments are host cells that can be regulated in their activity owing to genetic regulatory elements that are provided, for example, on the vector but can also be present in these cells from the outset. Expression in said cells may be induced, for example, by controlled addition of chemical compounds used as activators, by changing the cultivation conditions or when a particular cell density is reached. This allows economic production of the proteins according to the invention. An example of such a compound is IPTG, as described above.

Prokaryotic or bacterial cells are preferred host cells. Bacteria are characterized by short generation times and low demands on cultivation conditions. This makes it possible to establish cost-effective cultivation methods or production methods. In addition, a person skilled in the art will have a wealth of experience in the case of bacteria in fermentation technology. Gram-negative or gram-positive bacteria can be suitable for a specific production for many different reasons to be determined experimentally in each individual case, such as nutrient sources, product formation rate, time needed, etc.

In gram-negative bacteria, such as Escherichia coli, a plurality of proteins are secreted into the periplasmic space, i.e., into the compartment between the two membranes enclosing the cells. This can be advantageous for specific applications. Furthermore, gram-negative bacteria can also be designed such that they discharge the expressed proteins not only into the periplasmic space but into the medium surrounding the bacterium. Gram-positive bacteria such as bacilli or actinomycetes or other representatives of actinomycetes in contrast do not have an outer membrane, such that secreted proteins are immediately released into the medium surrounding the bacteria, usually the nutrient medium, from which the expressed proteins can be purified. They can be isolated directly from the medium or further processed. In addition, gram-positive bacteria are related or identical to most origin organisms for technically important enzymes and usually themselves form comparable enzymes, such that they have a similar codon usage and their protein synthesis apparatus is naturally aligned accordingly.

Host cells according to the invention may be altered in terms of their requirements for culture conditions, have different or additional selection markers, or also express different or additional proteins. In particular, this may also involve those host cells which express a plurality of proteins or enzymes.

The present invention can be applied in principle to all microorganisms, in particular to all fermentable microorganisms, particularly preferably to those of the genus Bacillus, and allows proteins according to the invention to be produced using such microorganisms. Such microorganisms then represent host cells for the purposes of the invention.

In a further embodiment of the invention, the host cell is characterized in that it is a bacterium, preferably one selected from the group of the genera of Escherichia, Klebsiella, Bacillus, Staphylococcus, Corynebacterium, Arthrobacter, Streptomyces, Stenotrophomonas and Pseudomonas, more preferably one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

However, the host cell can also be a eukaryotic cell that is characterized in that it has a nucleus. The invention therefore further relates to a host cell that is characterized in that it has a nucleus. In contrast with prokaryotic cells, eukaryotic cells are capable of post-translationally modifying the protein formed. Examples thereof are fungi, such as actinomycetes or yeasts, such as Saccharomyces or Kluyveromyces. This can be particularly advantageous, for example, if the proteins are to undergo specific modifications in connection with their synthesis, which modifications make such systems possible. Modifications carried out by eukaryotic systems, in particular in connection with the protein synthesis, include, for example, the binding of low-molecular-weight compounds such as membrane anchors or oligosaccharides. Such oligosaccharide modifications can be desirable, for example, to reduce the allergenicity of an expressed protein. Coexpression with the enzymes naturally formed by such cells, such as cellulases, can also be advantageous. Furthermore, for example, thermophilic fungal expression systems can be particularly suitable for expression of temperature-resistant proteins or variants.

The host cells according to the invention are cultured and fermented in the usual manner, for example in discontinuous or continuous systems. In the first case, a suitable nutrient medium is inoculated with the host cells and the product is harvested from the medium after a period to be determined experimentally. Continuous fermentations are characterized by achieving a flow equilibrium in which cells partially die off over a comparatively long period but also grow back and, at the same time, the protein formed can be removed from the medium.

Host cells according to the invention are preferably used to produce protease conjugates according to the invention. The invention therefore further relates to a method for producing a protease conjugate, comprising
(a) cultivating a host cell according to the invention, and
(b) isolating the protease conjugate from the culture medium or from the host cell.

This subject matter of the invention preferably comprises fermentation processes. Fermentation methods are known per se from the prior art and represent the actual large-scale production step, generally followed by a suitable purification method for the product produced, for example for the protease conjugates according to the invention. All fermentation methods that are based on a corresponding method for producing a protease conjugate according to the invention represent embodiments of this subject matter of the invention.

Fermentation processes that are characterized in that the fermentation is carried out via a feed strategy are considered in particular. In this case, the media constituents that are consumed by the continuous cultivation are added. As a result, considerable increases can be achieved both in the cell density and in the cell mass or dry mass and/or in particular in the activity of the enzyme of interest. Furthermore, the fermentation can also be designed such that unwanted metabolic products are filtered out or neutralized by adding buffers or suitable counterions.

The produced protease conjugate may be subsequently harvested from the fermentation medium. Such a fermentation method is preferred over isolation of the protease conjugate from the host cell, i.e., product preparation from the cell mass (dry mass), but requires the provision of suitable host cells or one or more suitable secretion markers or mechanisms and/or transport systems so that the host cells secrete the protease conjugate into the fermentation medium. Without secretion, the isolation of the protease conjugate from the host cell, i.e., purification thereof from the cell mass, can alternatively take place, for example by means of precipitation with ammonium sulfate or ethanol or by means of chromatographic purification.

The invention further provides an agent that is characterized in that it contains at least one protease conjugate as described and defined above. The agent is preferably a washing or cleaning agent.

Unless explicitly indicated otherwise, all percentages that are cited in connection with the compositions/agents described herein relate to wt. %, in each case based on the relevant mixture/the relevant agent.

Within the scope of the present invention, unless stated otherwise, fatty acids and/or fatty alcohols and/or the derivatives thereof represent branched or unbranched carboxylic acids and/or alcohols and/or the derivatives thereof preferably having 6 to 22 carbon atoms. In particular, the oxo-alcohols or derivatives thereof which can be obtained, for example, by Roelen's oxo synthesis can be used accordingly.

Whenever alkaline-earth metals are mentioned in the following as counterions for monovalent anions, this means that the alkaline-earth metal is naturally only present in half the amount of substance—sufficient to balance the charge—of the anion.

According to the invention, all conceivable types of washing or cleaning agents are to be understood as washing or cleaning agents, both concentrates and undiluted agents, for use on a commercial scale, in washing machines or for hand washing or cleaning. These include, for example, detergents for textiles, carpets or natural fibers, for which the term detergent is used. These also include, for example, dishwashing detergents for dishwashers (automatic dishwashing detergents) or manual dishwashing detergents or cleaners for hard surfaces such as metal, glass, porcelain, ceramics, tiles, stone, painted surfaces, plastics, wood or leather, for which the term cleaning agent is used, i.e., in addition to manual and automatic dishwashing detergents, also, for example, scouring agents, glass cleaners, WC rim blocks, etc. The washing and cleaning agents within the scope of the invention also include auxiliary washing agents which are added to the actual washing agent during manual or automatic textile washing in order to achieve a further effect. Furthermore, within the scope of the invention, washing and cleaning agents also include textile pre-treatment agents and post-treatment agents, e.g., agents with which the item of laundry is brought into contact before the actual washing, e.g., for dissolving stubborn soiling, and also agents which, in a step downstream of the actual textile washing, impart further desirable properties to the laundry item, such as a pleasant feel, crease resistance or low static charge. Inter alia, softeners are included in the latter agents.

The washing or cleaning agents according to the invention, which may be in the form of powdered or granular solids, in compacted or further-compacted particulate form, homogeneous solutions or suspensions, may contain, in addition to a protease according to the invention, all known ingredients conventional in such agents, with preferably at least one other ingredient being present in the agent. The agents according to the invention can in particular contain surfactants, builders, polymers, glass corrosion inhibitors, corrosion inhibitors, bleaching agents such as peroxygen compounds, bleach activators or bleach catalysts. They may also contain water-miscible organic solvents, further enzymes, enzyme stabilizers, sequestering agents, electrolytes, pH regulators and/or further auxiliaries, such as optical brighteners, graying inhibitors, dye transfer inhibitors, foam regulators, as well as dyes and fragrances, and combinations thereof.

Advantageous ingredients of agents according to the invention are disclosed in international patent application WO 2009/121725, starting at the penultimate paragraph of page 5 and ending after the second paragraph on page 13. Reference is expressly made to this disclosure and the disclosure therein is incorporated into the present patent application.

An agent according to the invention advantageously contains at least one protease conjugate according to the invention in an amount of 2 g to 20 mg, preferably of 5 g to 17.5 mg, particularly preferably of 20 g to 15 mg, and very particularly preferably of 50 g to 10 mg per g of the agent. In various embodiments, the concentration of the protease conjugate (active enzyme) described herein in the agent is >0 to 1 wt. %, preferably 0.0001 to 0.001 wt. %, more preferably 0.001 to 0.1 wt. %, based on the total weight of the agent.

An agent according to the invention contains the protease conjugate in an amount, increasingly preferably, of 1×10⁻⁸ to 5 wt. %, of 0.0001 to 1 wt. %, of 0.0005 to 0.5 wt. %, of 0.001 to 0.1 wt. %, in each case relative to active protein and relative to the total weight of the washing agent.

The embodiments of the present invention include all solid, powdered, liquid, gel or pasty administration forms of agents according to the invention, which may optionally also consist of a plurality of phases and can be present in compressed or uncompressed form. The agent can be present as a free-flowing powder, in particular having a bulk density of 300 g/l to 1200 g/1, in particular 500 g/l to 900 g/l or 600 g/l to 850 g/1. The solid administration forms of the agent further include extrudates, granules, tablets or pouches. Alternatively, the agent can also be in a liquid, gel or pasty form, e.g., in the form of a non-aqueous liquid washing agent or a non-aqueous paste or in the form of an aqueous liquid washing agent or an aqueous paste. Liquid agents are generally preferred. Furthermore, the agent can be present as a single-component system. Such agents consist of one phase. Alternatively, an agent can also consist of a plurality of phases. Such an agent is accordingly divided into a plurality of components.

If the washing agents according to the invention are in liquid form, they preferably contain more than 40 wt. %, preferably 50 to 90 wt. %, and particularly preferably 60 to 80 wt. %, water, relative to their total weight.

The agents according to the invention can contain one or more surfactants, with anionic surfactants, non-ionic surfactants, and mixtures thereof being particularly suitable, although cationic, zwitterionic, and/or amphoteric surfactants can also be contained. The agents preferably contain 5 to 70 wt. % surfactant, preferably 5 to 60 wt. %, and more preferably 5 to 50 wt. % surfactant.

Suitable anionic surfactants are, in particular, soaps and those which contain sulfate or sulfonate groups, preferably having alkali ions as cations. Usable soaps are preferably the alkali salts of saturated or unsaturated C₁₂₋₁₈ fatty acids. Fatty acids of this kind can also be used in a not completely neutralized form. Suitable sulfate-type surfactants include the salts of sulfuric acid half-esters of C₁₂₋₁₈ fatty alcohols atoms and the sulfation products of the mentioned non-ionic surfactants having a low degree of ethoxylation. Surfactants of the sulfonate type that can be used include, for example, C₉₋₁₄ alkylbenzene sulfonates, alkane sulfonates obtained from C₁₂₋₁₈ alkanes, for example by sulfochlorination or sulfoxidation with subsequent hydrolysis or neutralization, C₁₂₋₁₈ olefin sulfonates resulting from the reaction of corresponding monoolefins with sulfur trioxide, mixtures of alkene and hydroxyalkane sulfonates, disulfonates, such as those obtained from C₁₂₋₁₈ monoolefins with terminal or internal double bonds by sulfonation with gaseous sulfur trioxide and subsequent alkaline or acidic hydrolysis of the sulfonation products, and ca-sulfofatty acid esters (ester sulfonates) resulting from the sulfonation of fatty acid methyl or ethyl esters, e.g., c-sulfonated methyl esters of hydrogenated coconut, palm kernel or tallow fatty acids.

The agent preferably comprises 2 to 55 wt. %, and more preferably 3 to 35 wt. %, anionic surfactant. Very particularly preferably, the agent comprises 3 to 25 wt. % alkylbenzene sulfonate. In addition, the agent can preferably also contain other anionic surfactants, in particular alkyl ether sulfates, and non-ionic surfactants, in particular fatty alcohol alkoxylates. These can then make up the remainder of the surfactants.

Suitable alkylbenzene sulfonates are preferably selected from linear or branched alkylbenzene sulfonates of the formula

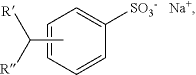

in which R′ and R″ are, independently of one another, hydrogen or alkyl, and together contain 6 to 19, preferably 7 to 15, and in particular 9 to 13, C atoms. A very particularly preferred representative is sodium dodecylbenzene sulfonate.

The alkali salts and in particular the sodium salts of the sulfuric acid half-esters of C₁₂₋₁₈ fatty alcohols, e.g. from coconut fatty alcohol, tallow fatty alcohol, lauryl alcohol, myristyl alcohol, cetyl alcohol or stearyl alcohol, or of C₁₀₋₂₀ oxo alcohols and the half-esters of secondary alcohols having these chain lengths are preferred as alk(en)yl sulfates. Alk(en)yl sulfates of the mentioned chain length that contain a synthetic straight-chain alkyl functional group prepared on a petrochemical basis and have a degradation behavior similar to that of the adequate compounds based on fat chemical raw materials are also preferred. From a washing perspective, the C₁₂₋₁₆ alkyl sulfates and C₁₂₋₁₅ alkyl sulfates and also C₁₄₋₁₅ alkyl sulfates are preferred.

The sulfuric acid monoesters of straight-chain or branched C₇₋₂₁ alcohols ethoxylated with 1 to 6 mol ethylene oxide, such as 2-methyl-branched C₉₋₁₁ alcohols with, on average, 3.5 mol ethylene oxide (EO) or C₁₂₋₁₈ fatty alcohols with 1 to 4 EO, are also suitable.

Suitable alkyl ether sulfates are, for example, compounds of the formula

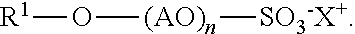

In this formula, R¹ represents a linear or branched, substituted or unsubstituted alkyl functional group, preferably a linear, unsubstituted alkyl functional group, particularly preferably a fatty alcohol functional group. Preferred functional groups R¹ are selected from decyl, undecyl, dodecyl, tridecyl, tetradecyl, pentadecyl, hexadecyl, heptadecyl, octadecyl, nonadecyl, eicosyl functional groups and mixtures thereof, the representatives having an even number of C atoms being preferred. Particularly preferred functional groups R¹ are derived from C₁₂₋₁₈ fatty alcohols, for example from coconut fatty alcohol, tallow fatty alcohol, lauryl, myristyl, cetyl or stearyl alcohol or from C₁₀₋₂₀ oxo alcohols. AO represents an ethylene oxide (EO) group or propylene oxide (PO) group, preferably an ethylene oxide group. The index n represents an integer from 1 to 50, preferably from 1 to 20, and in particular from 2 to 10. Very particularly preferably, n represents the numbers 2, 3, 4, 5, 6, 7 or 8. X⁺ represents a monovalent cation or the n-th part of an n-valent cation, the alkali metal ions, including Na⁺ or K⁺, being preferred in this case, with Na⁺ being most preferred. Further cations X⁺ may be selected from NH₄⁺, ½ Zn²⁺, ½ Mg²⁺, ½ Ca²⁺, ½ Mn²⁺, and the mixtures thereof.

In various embodiments, the alkyl ether sulfate can be selected from fatty alcohol ether sulfates of the formula

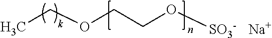

where k=11 to 19, and n=2, 3, 4, 5, 6, 7 or 8. Very particularly preferred representatives are Na—C₁₂₋₁₄ fatty alcohol ether sulfates having 2 EO (k=11-13, n=2). The degree of ethoxylation indicated represents a statistical average value which can be an integer or a fractional number for a specific product. The degrees of alkoxylation indicated represent statistical averages which can be an integer or a fractional number for a specific product. Preferred alkoxylates/ethoxylates have a narrowed homolog distribution (narrow range ethoxylates, NRE).

It has proven advantageous for cold-wash performance if the washing agents additionally contain soap(s). Preferred washing agents are therefore characterized in that they contain soap(s). Saturated fatty acid soaps are suitable, such as the salts of lauric acid, myristic acid, palmitic acid, stearic acid, hydrogenated erucic acid and behenic acid, and in particular soap mixtures derived from natural fatty acids, such as coconut fatty acids, palm kernel fatty acids or tallow fatty acids.

Suitable non-ionic surfactants are in particular alkyl glycosides and ethoxylation and/or propoxylation products of alkyl glycosides or linear or branched alcohols each having 8 to approximately 18 C atoms in the alkyl portion and 3 to 20, preferably 4 to 10, alkyl ether groups. Furthermore, corresponding ethoxylation and/or propoxylation products of N-alkylamines, vicinal diols, fatty acid esters, and fatty acid amides, which correspond to the above-mentioned long-chain alcohol derivatives with respect to the alkyl moiety, and of alkylphenols having 5 to 12 C atoms in the alkyl radical, may be used.

Non-ionic surfactants that are preferably used are alkoxylated, advantageously ethoxylated, in particular primary alcohols having preferably 8 to 18 C atoms and, on average, 1 to 12 mol ethylene oxide (EO) per mol of alcohol, in which the alcohol functional group can be linear or preferably methyl-branched in the 2-position, or can contain linear and methyl-branched functional groups in the mixture, as are usually present in oxo alcohol functional groups. However, alcohol ethoxylates having linear functional groups of alcohols of native origin having 12 to 18 C atoms, for example of coconut, palm, tallow fatty or oleyl alcohol, and an average of 2 to 8 EO per mol of alcohol, are particularly preferred. Preferred ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO or 8 EO, C₁₂₋₁₈ alcohols having 3 EO, 5 EO or 7 EO, and mixtures thereof, such as mixtures of C₁₂₋₁₄ alcohol having 3 EO and C₁₂₋₁₈ alcohol having 5 EO. The degrees of ethoxylation specified represent statistical averages that can correspond to an integer or a fractional number for a specific product. Preferred alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

Another class of non-ionic surfactants that are preferably used, which are used either as the sole non-ionic surfactant or in combination with other non-ionic surfactants, is alkoxylated, preferably ethoxylated or ethoxylated and propoxylated fatty acid alkyl esters, preferably having 1 to 4 carbon atoms in the alkyl chain, in particular fatty acid methyl esters.

Another class of non-ionic surfactants that can advantageously be used is the alkyl polyglycosides (APG). Usable alkyl polyglycosides satisfy the general formula

RO(G)_{z},
in which R is a linear or branched, in particular methyl-branched in the 2-position, saturated or unsaturated, aliphatic functional group having 8 to 22, preferably 12 to 18 C atoms and G is the symbol which represents a glycose unit having 5 or 6 C atoms, preferably glucose. The degree of glycosidation z is between 1 and 4, preferably between 1 and 2, and in particular between 1.1 and 1.4. Linear alkyl polyglycosides, i.e., alkyl polyglycosides in which the polyglycol functional group is a glucose functional group and the alkyl functional group is an n-alkyl functional group, are preferably used.

Non-ionic surfactants of the aminoxide type, for example N-cocoalkyl-N,N-dimethylamine oxide and N-tallow-alkyl-N,N-dihydroxyethylamine oxide, and of the fatty acid alkanolamides may also be suitable. The quantity of these non-ionic surfactants is preferably no more than that of the ethoxylated fatty alcohols, in particular no more than half thereof.

Suitable amphoteric surfactants are, for example, betaines of the formula

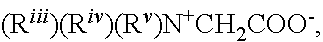

in which Rⁱⁱⁱ denotes an alkyl group, which is optionally interrupted by heteroatoms or heteroatom groups, having 8 to 25, preferably 10 to 21, carbon atoms, and R^{iv} and R^{v} denote identical or different alkyl functional groups having 1 to 3 carbon atoms, in particular C₁₀₋₁₈ alkyl dimethyl carboxymethyl betaine and C₁₁₁₇ alkyl amidopropyl dimethyl carboxymethyl betaine.

Suitable cationic surfactants include, inter alia, the quaternary ammonium compounds of the formula

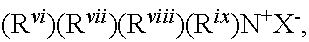

in which R^{vi} to R^{ix} represent four identical or different, in particular two long-chain and two short-chain, alkyl functional groups, and X⁻ represents an anion, in particular a halide ion, e.g., didecyldimethylammonium chloride, alkylbenzyldidecylammonium chloride, and mixtures thereof. Further suitable cationic surfactants are the quaternary surface-active compounds, in particular having a sulfonium, phosphonium, iodonium or arsonium group, which are also known as antimicrobial active ingredients. By using quaternary surface-active compounds having an antimicrobial effect, the agent can be provided with an antimicrobial effect or the antimicrobial effect that may already be present due to other ingredients can be improved.

Complexing agents are another preferred component of washing agents according to the invention. Particularly preferred complexing agents are the phosphonates, provided that their use is permitted by regulations. In addition to 1-hydroxyethane-1,1-diphosphonic acid, the complexing phosphonates include a number of different compounds such as diethylenetriamine penta(methylene phosphonic acid) (DTPMP). Hydroxyalkane or aminoalkane phosphonates are particularly preferred in this application. Among the hydroxyalkane phosphonates, 1-hydroxyethane-1,1-diphosphonate (HEDP) is of particular importance as a cobuilder. It is preferably used as a sodium salt, the disodium salt reacting neutrally and the tetrasodium salt reacting alkaline (pH 9). Possible amino alkane phosphonates preferably include ethylenediamine tetramethylene phosphonate (EDTMP), diethylenetriamine pentamethylene phosphonate (DTPMP) and the higher homologs thereof. They are preferably used in the form of the neutrally reacting sodium salts, e.g., as hexasodium salt of EDTMP or as the heptasodium and octasodium salt of DTPMP. Of the class of phosphonates, HEDP is preferably used as a builder. The aminoalkane phosphonates also have a pronounced heavy metal binding capacity. Accordingly, it may be preferred, in particular if the agents also contain bleach, to use aminoalkane phosphonates, in particular DTPMP, or to use mixtures of the aforementioned phosphonates. A preferred washing agent in the context of this application contains one or more phosphonate(s) from the group aminotrimethylene phosphonic acid (ATMP) and/or the salts thereof; ethylenediamine tetra(methylene phosphonic acid) (EDTMP) and/or the salts thereof; diethylenetriamine penta(methylene phosphonic acid) (DTPMP) and/or the salts thereof; 1-hydroxyethane-1,1-diphosphonic acid (HEDP) and/or the salts thereof; 2-phosphonobutane-1,2,4-tricarboxylic acid (PBTC) and/or the salts thereof; hexamethylenediamine tetra(methylene phosphonic acid) (HDTMP) and/or the salts thereof; nitrilotri(methylenephosphonic acid) (NTMP) and/or the salts thereof. Washing agents which contain 1-hydroxyethane-1,1-diphosphonic acid (HEDP) or diethylenetriaminepenta(methylenephosphonic acid) (DTPMP) as phosphonates are particularly preferred. The washing agents according to the invention may, of course, contain two or more different phosphonates. Washing agents that are preferred according to the invention are characterized in that the washing agent contains at least one complexing agent from the group of phosphonates, preferably 1-hydroxyethane-1,1-diphosphonate, the proportion by weight of the phosphonate with respect to the total weight of the washing agent preferably being between 0.1 and 8.0 wt. %, more preferably 0.2 and 5.0 wt. %, even more preferably 0.3 and 3.0 wt. %, and particularly preferably 0.5-2.0 wt. %.

The washing agents according to the invention also preferably contain builders, preferably at least one water-soluble and/or water-insoluble, organic, and/or inorganic builder. The builders include in particular the silicates, carbonates and/organic cobuilders.

Polycarboxylates/polycarboxylic acids, polymeric polycarboxylates, aspartic acid, polyacetals, dextrins, other organic cobuilders and phosphonates are particularly noteworthy as organic cobuilders. These substance classes are described below. Organic cobuilder substances of this kind can, if desired, be contained in amounts of up to 40 wt. %, in particular up to 25 wt. %, and preferably of 1 to 8 wt. %. Suitable organic builder substances are, for example, the polycarboxylic acids that can be used in the form of the free acids and/or the sodium salts thereof, where polycarboxylic acids are understood to mean the carboxylic acids which carry more than one acid function. For example, these are citric acid, adipic acid, succinic acid, glutaric acid, malic acid, tartaric acid, maleic acid, fumaric acid, saccharic acid and carboxymethyl inulines, monomeric and polymeric aminopolycarboxylic acids, in particular glycine diacetic acid, methylglycine diacetic acid, nitrilotriacetic acid (NTA), iminodisuccinates such as ethylenediamine-N,N′-disuccinic acid and hydroxyiminodisuccinates, ethylenediaminetetraacetic acid and polyaspartic acid, polyphosphonic acids, in particular aminotris(methylenephosphonic acid), ethylenediamine tetrakis(methylenephosphonic acid), lysine tetra(methylenephosphonic acid) and 1-hydroxyethane-1,1-diphosphonic acid, polymeric hydroxy compounds such as dextrin, and polymeric (poly)carboxylic acids, polycarboxylates which can be obtained in particular by oxidizing polysaccharides or dextrins, and/or polymeric acrylic acids, methacrylic acids, maleic acids, and mixed polymers thereof, which may also contain small amounts of polymerizable substances without carboxylic acid functionality in polymerized form. Organic builder substances of this kind can, if desired, be contained in amounts of up to 50 wt. %, in particular up to 25 wt. %, preferably of from 10 to 20 wt. % and particularly preferably from 1 to 5 wt. %. In addition to their builder effect, the free acids typically also have the property of being an acidification component and are thus also used for setting a lower and milder pH of washing agents. Particularly noteworthy here are citric acid, succinic acid, glutaric acid, adipic acid, gluconic acid, and any mixtures thereof. Citric acid or salts of citric acid are particularly preferably used as builder substances. Further particularly preferred builder substances are selected from methylglycinediacetic acid (MGDA), glutamic acid diacetate (GLDA), aspartic acid diacetate (ASDA), hydroxyethyl iminodiacetate (HEIDA), iminodisuccinate (IDS), ethylenediamine disuccinate (EDDS), carboxymethyl inulin and polyaspartate. In preferred embodiments, citric acid and/or citrate is used as the water-soluble, organic builder. It is particularly preferred to use 0.5 to 25 wt. %, preferably 0.75 to 12.5 wt. %, more preferably 1 to 4 wt. %, citric acid and/or 0.5 to 25 wt. %, preferably 0.75 to 12.5 wt. %, more preferably 1 to 4 wt. %, citrate, preferably alkali citrate, more preferably sodium citrate. Citric acid/citrate can each be used in the form of their hydrates, for example citric acid can be used in the form of the monohydrate, and citrate can be used in the form of the trisodium citrate dihydrate.

Polymeric polycarboxylates are also suitable as builders, e.g., the alkali metal salts of polyacrylic acid or polymethacrylic acid, e.g., those with a relative molecular mass of 500 to 70,000 g/mol. For the purpose of this application, the molar masses indicated for polymeric polycarboxylates are weight-average molar masses Mw of the respective acid form which have been determined in principle using gel permeation chromatography (GPC), a UV detector having been used. The measurement was carried out against an external polyacrylic acid standard which, due to the structural relationship to the tested polymers, yields realistic molecular weight values. These specifications differ significantly from the molecular weight specifications for which polystyrene sulfonic acids are used as the standard. The molar masses measured against polystyrene sulfonic acids are generally considerably higher than the molar masses indicated in this application. Suitable polymers are in particular polyacrylates which preferably have a molecular mass of from 2,000 to 20,000 g/mol. Due to their superior solubility, short-chain polyacrylates having molar masses of from 2,000 to 10,000 g/mol, and particularly preferably of from 3,000 to 5,000 g/mol, may in turn be preferred from this group. In addition, copolymeric polycarboxylates are suitable, in particular those of acrylic acid with methacrylic acid and those of acrylic acid or methacrylic acid with maleic acid. Copolymers of acrylic acid with maleic acid containing 50 to 90 wt. % acrylic acid and 50 to 10 wt. % maleic acid have proven to be particularly suitable. The relative molecular mass thereof, based on free acids, is generally 2,000 to 70,000 g/mol, preferably 20,000 to 50,000 g/mol, and in particular 30,000 to 40,000 g/mol.

A solid agent according to the invention preferably contains at least one water-soluble and/or water-insoluble, organic and/or inorganic builder. The water-soluble organic builder substances include the aforementioned organic builder substances.

In addition to the water-soluble organic builders mentioned above, the agents of the invention may also further contain inorganic water-soluble builders. In particular, alkali silicates, alkali carbonates, alkali hydrogen carbonates, alkali phosphates and/or sesquicarbonates, which can be present in the form of their alkaline, neutral, or acidic sodium or potassium salts, can be used as water-soluble inorganic builder materials. Small amounts of calcium carbonate may optionally also be contained in solid textile washing agents. Water-soluble crystalline and/or amorphous alkali silicates are suitable, for example. The alkali silicates that can be used in the agents according to the invention as builders preferably have a molar ratio of alkali oxide to SiO₂ of less than 0.95, in particular of from 1:1.1 to 1:12, and may be present in amorphous or crystalline form. Preferred alkali silicates are sodium silicates, in particular amorphous sodium silicates, having a molar ratio of Na₂O:SiO₂ of from 1:2 to 1:2.8. As crystalline silicates, which can be present alone or in a mixture with amorphous silicates, crystalline phyllosilicates of the general formula Na₂SiₓO₂ₓ₊₁·y H₂O are preferably used, in which x, known as the modulus, is a number from 1.9 to 22, in particular 1.9 to 4, and y is a number from 0 to 33, and preferred values for x are 2, 3 or 4. Preferred crystalline phyllosilicates are those in which x assumes the values 2 or 3 in the mentioned general formula. In particular, both β- and δ-sodium disilicates (Na₂Si₂O₅·y H₂O) are preferred. Practically water-free crystalline alkali silicates of the above general formula, in which x is a number from 1.9 to 2.1 and which are produced from amorphous alkali silicates, may also be used in agents according to the invention. In a further embodiment of agents according to the invention, a crystalline sodium phyllosilicate having a module of from 2 to 3, as can be prepared from sand and soda, is used. Crystalline sodium silicates having a module in the range of from 1.9 to 3.5 are used in a further embodiment of agents according to the invention. In agents containing amorphous and crystalline alkali silicates, the weight ratio of amorphous alkali silicate to crystalline alkali silicate is preferably from 1:2 to 2:1 and in particular from 1:1 to 2:1. Crystalline phyllosilicates of the above formula (I) are sold by Clariant GmbH under the trade name Na-SKS—for example, Na-SKS-1 (Na₂Si₂₂O₄₅·x H₂O, kenyaite), Na-SKS-2 (Na₂Si₁₄O₂₉·x H₂O, magadiite), Na-SKS-3 (Na₂Si₈O₇·x H₂O), or Na-SKS-4 (Na₂Si₄O₉·x H₂O, macatite). Of these, Na-SKS-5 (α-Na₂Si₂O₅), Na-SKS-7 (β-Na₂Si₂O₅, natrosilite), Na-SKS-9 (NaHSi₂O₅·3 H₂O), Na-SKS-10 (NaHSi₂O₅·3 H₂O, kanemite), Na-SKS-11 (t-Na₂Si₂O₅) and Na-SKS-13 (NaHSi₂O₅), but in particular Na-SKS-6 (6-Na₂Si₂O₅) are particularly suitable. In one embodiment of agents according to the invention, a granular compound made of crystalline phyllosilicate and citrate, crystalline phyllosilicate and the above-described (co)polymeric polycarboxylic acid, or alkali silicate and alkali carbonate is used, as is commercially available under the name Nabion® 15, for example. Water-soluble inorganic builder materials of this kind are contained in agents according to the invention preferably in amounts of from 1 to 20 wt. %, in particular 5 to 15 wt. %. Also of significance as water-soluble inorganic builder substances are the carbonates (and hydrogen carbonates), in particular sodium carbonate, and the phosphonic acids/phosphonates.

The agents according to the invention are preferably free of phosphate builders, i.e., they contain less than 1 wt. %, and preferably no phosphate builders are added intentionally.

The agents can also contain water-insoluble builders. Crystalline or amorphous water-dispersible alkali aluminosilicates, in amounts of up to 50 wt. %, preferably not more than 40 wt. %, in particular of from 3 to 20 wt. % and particularly preferably from 1 to 15 wt. %, are used in particular as water-insoluble inorganic builder materials. Among these, the crystalline sodium aluminosilicates in washing agent quality, in particular zeolite A, zeolite P, zeolite MAP and optionally zeolite X, either alone or in mixtures, for example in the form of a co-crystallizate of the zeolites A and X (Vegobond® AX, a commercial product from Condea Augusta S.p.A.), are preferred. Amounts close to the stated upper limit are preferably used in solid, particulate agents. Suitable aluminosilicates have, in particular, no particles having a particle size above 30 μm and preferably consist by at least 80 wt. % of particles having a size below 10 μm. The calcium binding capacity, which can be determined according to DE 2412837 A1, of said aluminosilicates is generally in the range of from 100 to 200 mg CaO per gram.

In addition to the previously described builders, polymers having a cleaning action can be contained in the washing agent. The proportion by weight of the polymers with respect to the total weight of washing agents according to the invention is preferably 0.1 to 20 wt. %, more preferably 1.0 to 15 wt. %, and even more preferably 2.0 to 12 wt. %.

Possible peroxygen compounds suitable for use in the agents according to the invention include, in particular, organic peroxy acids or peracid salts of organic acids, such as phthalimidopercaproic acid, perbenzoic acid, or salts of diperdodecanedioic acid, hydrogen peroxide and inorganic salts releasing hydrogen peroxide under the washing conditions, which salts include perborate, percarbonate, persilicate, and/or persulfates such as caroate, as well as hydrogen peroxide inclusion compounds such as H₂O₂-urea adducts. Hydrogen peroxide can also be produced by means of an enzymatic system, i.e., an oxidase and the substrate thereof. If solid peroxygen compounds are intended to be used, these may be used in the form of powders or granules, which may also be coated in a manner known in principle. The peroxygen compounds can be added to the washing liquor as such or in the form of the agents containing them, which in principle can contain all conventional washing, cleaning or disinfectant components. Particularly preferably, alkali percarbonate or alkali perborate monohydrate is used. If an agent according to the invention contains peroxygen compounds, these are present in amounts of preferably up to 50 wt. %, in particular of 5 to 30 wt. %, and more preferably of 0.1 to 20 wt. %.

Compounds which, under perhydrolysis conditions, result in aliphatic peroxocarboxylic acids having preferably 1 to 10 C atoms, in particular 2 to 4 C atoms, and/or optionally substituted perbenzoic acid, may be used in the agents as bleach activators. Suitable substances are those which carry O- and/or N-acyl groups of the stated number of C atoms and/or optionally substituted benzoyl groups. Preferred are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenol sulfonates or carboxylates or the sulfonic or carboxylic acids thereof, in particular nonanoyloxybenzenesulfonate or isononanoyloxybenzenesulfonate or laroyloxybenzenesulfonate (NOBS or iso-NOBS or LOBS), 4-(2-decanoyloxyethoxycarbonyloxy)-benzenesulfonate (DECOBS) or decanoyloxybenzoate (DOBA), carboxylic acid anhydrides, in particular phthalic acid anhydride, acylated polyhydric alcohols, in particular triacetin, ethylene glycol diacetate, 2,5-diacetoxy-2,5-dihydrofuran and enol esters, as well as acetylated sorbitol and mannitol or the described mixtures thereof (SORMAN), acylated sugar derivatives, in particular pentaacetyl glucose (PAG), pentaacetyl fructose, tetraacetylxylose and octaacetyl lactose, acetylated, optionally N-alkylated glucamine and gluconolactone, N-acylated lactams, for example N-benzoylcaprolactam, nitriles from which perimidic acids are formed, in particular aminoacetonitrile derivatives having a quaternized nitrogen atom, and/or oxygen-transferring sulfonimines and/or acylhydrazones. The hydrophilically substituted acyl acetals and the acyl lactams are likewise preferably used. Combinations of conventional bleach activators can also be used. Such bleach activators can, in particular in the presence of the above-mentioned hydrogen peroxide-yielding bleaching agents, be present in the customary quantity range, preferably in amounts of from 0.5 to 10 wt. %, and in particular 1 to 8 wt. %, based on the total agent, but are preferably entirely absent when percarboxylic acid is used as the sole bleaching agent.

In addition to or instead of the conventional bleach activators, sulfonimines and/or bleach-boosting transition metal salts or transition metal complexes may also be contained in solid agents as what are referred to as bleach catalysts.

Suitable graying inhibitors or soil release active ingredients (soil release polymer) are cellulose ethers, such as carboxymethyl cellulose, methyl cellulose, hydroxyalkyl celluloses and mixed cellulose ethers, such as methyl hydroxyethyl cellulose, methylhydroxypropyl cellulose and methyl carboxymethyl cellulose. Sodium carboxymethyl cellulose, hydroxypropyl methyl cellulose and mixtures thereof and, optionally, mixtures thereof with methyl cellulose are preferably used. The soil release active ingredients commonly used include copolyesters containing dicarboxylic acid units, alkylene glycol units and polyalkylene glycol units. The proportion of graying inhibitors and/or soil release active ingredients in agents according to the invention is generally no greater than 2 wt. % and is preferably 0.5 to 1.5 wt. %, particularly preferably 0.5 to 2 wt. %.

Derivatives of diaminostilbene disulfonic acid or the alkali metal salts thereof can be contained, for example, as optical brighteners, in particular for textiles made of cellulose fibers (e.g., cotton). Salts of 4,4′-bis(2-anilino-4-morpholino-1,3,5-triazinyl-6-amino)stilbene-2,2′-disulfonic acid or compounds having a similar structure which, instead of the morpholino group, have a diethanolamino group, a methylamino group, or a 2-methoxyethylamino group are suitable, for example. Furthermore, brighteners of the substituted 4,4′-distyryl-diphenyl type can be present, e.g., 4,4′-bis-(4-chloro-3-sulfostyryl)-diphenyl. Mixtures of brighteners can also be used. Brighteners of the 1,3-diaryl-2-pyrazoline type, e.g., 1-(p-sulfoamoylphenyl)-3-(p-chlorophenyl)-2-pyrazoline, and compounds having a similarly structure are particularly suitable for polyamide fibers. The content of optical brighteners or brightener mixtures in the agent is generally no greater than 1 wt. %, preferably 0.05 to 0.5 wt. %. In a preferred embodiment of the invention, the agent is free of such active ingredients.

The customary foam regulators that can be used in the agents according to the invention include, for example, polysiloxane-silicic acid mixtures, the finely divided silicic acid contained therein preferably being silanized or otherwise hydrophobized. The polysiloxanes can consist of both linear compounds and crosslinked polysiloxane resins and mixtures thereof. Further defoamers are paraffinic hydrocarbons, in particular microparaffins and paraffin waxes of which the melting point is above 40° C., saturated fatty acids or soaps having in particular 20 to 22 C atoms, for example sodium behenate, and alkali salts of phosphoric acid mono- and/or dialkyl esters, in which the alkyl chains each have 12 to 22 C atoms. Among these, sodium monoalkyl phosphate and/or dialkyl phosphate having C₁₆₋₁₈ alkyl groups is preferably used. The proportion of foam regulators can preferably be 0.2 to 2 wt. %, particularly preferably not more than 1 wt. %.

In order to set the desired pH, the agents according to the invention can contain acids that are compatible with the system and environment, in particular citric acid, acetic acid, tartaric acid, malic acid, lactic acid, glycolic acid, succinic acid, glutaric acid, and/or adipic acid, but also mineral acids, in particular sulfuric acid or alkali hydrogen sulfates, or bases, in particular ammonium or alkali hydroxides, preferably sodium hydroxide. These types of pH regulators are contained in the agents according to the invention in amounts preferably no greater than 10 wt. %, in particular of from 0.5 to 6 wt. %, particularly preferably from 0.3 to 2 wt. %.

The washing agents according to the invention can contain an organic solvent as a further component. Adding organic solvents has an advantageous effect on the enzyme stability and cleaning performance of these agents. Preferred organic solvents are derived from the group of monohydric or polyhydric alcohols, alkanolamines or glycol ethers. The solvents are preferably selected from ethanol, n- or i-propanol, butanol, glycol, propanediol, butanediol, glycerol, diglycol, propylene diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol methyl ether, dipropylene glycol ethyl ether, methoxytriglycol, ethoxytriglycol, butoxytriglycol, 1-butoxyethoxy-2-propanol, 3-methyl-3-methoxybutanol, propylene-glycol-t-butyl ether and mixtures of these solvents. The proportion by weight of these organic solvents with respect to the total weight of the washing agents according to the invention is preferably 0.1 to 10 wt. %, more preferably 0.2 to 8.0 wt. %, and even more preferably 0.5 to 5.0 wt. %. A particularly preferred organic solvent which is particularly effective in stabilizing the washing agents is glycerol, as well as 1,2-propylene glycol. Liquid washing agents preferably comprise at least one polyol, preferably from the group glycerol and 1,2-propylene glycol, relative to the total weight of the washing agent, preferably in an amount of 0.1 to 10 wt. %, preferably 0.2 to 8.0 wt. %, and more preferably 0.5 to 5.0 wt. %. Other preferred organic solvents are the organic amines and alkanolamines. The washing agents according to the invention preferably contain these amines in amounts of 0.1 to 10 wt. %, more preferably of 0.2 to 8.0 wt. %, and even more preferably of 0.5 to 5.0 wt. %, in each case relative to the total weight thereof. Ethanolamine is a particularly preferred alkanolamine.

Washing or cleaning agents according to the invention can contain only one protease in the form of a protease conjugate according to the invention. Alternatively, they can also contain further hydrolytic enzymes or other enzymes in a concentration expedient for the effectiveness of the agent. A further embodiment of the invention is thus represented by agents that further comprise one or more further enzymes. Further enzymes which can preferably be used are all enzymes which can exhibit catalytic activity in the agent according to the invention, in particular a lipase, amylase, cellulase, hemicellulase, mannanase, tannase, xylanase, xanthanase, xytoglucanase, β-glucosidase, pectinase, carrageenase, perhydrolase, oxidase, oxidoreductase, as well as other protease, i.e., proteases that are not present in the form of a conjugate according to the invention, as well as mixtures thereof. Further enzymes are advantageously contained in the agent in each case in an amount of 1×10⁻⁸ to 5 wt. %, based on the active protein. Increasingly preferably, each further enzyme is contained in agents according to the invention in an amount of from 1×10⁻⁷ to 3 wt. %, from 0.00001 to 1 wt. %, from 0.00005 to 0.5 wt. %, from 0.0001 to 0.1 wt. % and particularly preferably from 0.0001 to 0.05 wt. %, based on active protein. Particularly preferably, the enzymes exhibit synergistic cleaning performance with respect to particular dirt or stains, i.e. the enzymes contained in the agent composition assist one another in their cleaning performance. Synergism of this kind is very particularly preferably present between the protease conjugate contained according to the invention and a further enzyme of an agent according to the invention, including in particular between the protease conjugate according to the invention and an amylase and/or a lipase and/or a mannanase and/or a cellulase and/or a pectinase. Synergistic effects can occur not only between different enzymes but also between one or more enzymes and other ingredients of the agent according to the invention.

Textile washing agents preferred according to the invention have at least one protease conjugate and at least one amylase. In a further preferred embodiment of the invention, textile washing agents have at least one protease conjugate and at least one cellulase. In a further preferred embodiment, textile washing agents have at least one protease conjugate, and at least one lipase. In a further preferred embodiment, textile washing agents have at least one protease conjugate, at least one amylase, and at least one lipase. In a further preferred embodiment, textile washing agents have at least one protease conjugate, at least one amylase, and at least one cellulase. In a further preferred embodiment, textile washing agents have at least one protease conjugate, at least one amylase, at least one cellulase, and at least one lipase. Textile washing agents which have 3 to 10 different enzymes are particularly preferred, it being possible for textile washing agents which have 3 to 10 different types of enzymes to be particularly preferred with regard to the cleaning performance over a very broad spectrum of stains.

Examples of proteases which are used in agents according to the invention in addition to the at least one protease conjugate as defined and described here are the subtilisins BPN′ from Bacillus amyloliquefaciens and Carlsberg from Bacillus licheniformis, protease PB92, subtilisins 147 and 309, the protease from Bacillus lentus, subtilisin DY, and the enzymes thermitase, proteinase K and proteases TW3 and TW7, which in the narrower sense are associated with the subtilases but no longer with the subtilisins. Subtilisin Carlsberg is available in a developed form under the trade name Alcalase® from Novozymes. Subtilisins 147 and 309 are marketed by Novozymes under the trade names Esperase® and Savinase®, respectively. The protease variants are derived from the protease from Bacillus lentus DSM 5483. Other proteases that are suitable are, for example, the enzymes available under the trade names Durazym®, Relase®, Everlase®, Nafizym®, Natalase®, Kannase®, Progress Uno 101L® and Ovozyme® from Novozymes, the enzymes available under the trade names Purafect®, Purafect® OxP, Purafect® Prime, Excellase® and Properase®, Preferenz P100® and Preferenz P300® from Danisco/DuPont, the enzyme available under the trade name Lavergy pro 104 LS® from BASF, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease P® from Amano Pharmaceuticals Ltd., and the enzyme available under the name Proteinase K-16 from Kao Corp. The proteases from Bacillus gibsonii and Bacillus pumilus, which are disclosed in WO 2008/086916, WO 2007/131656, WO 2017/215925, WO 2021/175696 and WO 2021/175697, are particularly preferably used. Further proteases which can be used advantageously are disclosed in e.g., WO 91/02792, WO 2008/007319, WO 93/18140, WO 01/44452, GB 1243784 A, WO 96/34946, WO 02/029024, and WO 03/057246. Further proteases that can be used are those which are naturally present in the microorganisms Stenotrophomonas maltophilia, in particular Stenotrophomonas maltophilia K279a, Bacillus intermedius and Bacillus sphaericus.

Examples of amylases are the a-amylases from Bacillus licheniformis, Bacillus amyloliquefaciens or Bacillus stearothermophilus, as well as in particular the developments thereof that have been improved for use in washing or cleaning agents. The enzyme from Bacillus licheniformis is available from Novozymes under the name Termamyl® and from Danisco/DuPont under the name Purastar® ST. Development products of this α-amylase are available under the trade names Duramyl® and Termamyl® ultra (both from Novozymes), Purastar® OxAm (Danisco/DuPont) and Keistase® (Daiwa Seiko Inc.). The (α-amylase from Bacillus amyloliquefaciens is marketed by Novozymes under the name BAN®, and derived variants from the α-amylase from Bacillus stearothermophilus are marketed under the names BSG® and Novamyl®, also by Novozymes. Others that are particularly noteworthy for this purpose are the α-amylases from Bacillus sp. A 7-7 (DSM 12368) and the cyclodextrin glucanotransferase (CGTase) from Bacillus agaradhaerens (DSM 9948) should be emphasized. Furthermore, the amylolytic enzymes which are disclosed in WO 95/26397, WO 96/23873, WO 99/23211, WO 00/60060, WO 2003/002711, WO 2003/054177, WO 2006/002643, WO 2007/079938, WO 2011/100410, and WO 2013/003659 can be used. Fusion products of all mentioned molecules can also be used. Furthermore, the developments of the α-amylase from Aspergillus niger and A. oryzae, available under the trade name Fungamyl® from Novozymes, are suitable. Other commercial products that can be advantageously used are, for example, Amylase-LT® and Stainzyme® or Stainzyme® ultra or Stainzyme® plus as well as Amplify™ 12L or Amplify Prime™ 100L, the latter also from Novozymes, and the PREFERENZ S® series from Danisco/DuPont, including, for example, PREFERENCE S100@, PREFERENCE S1000® or PREFERENCE S210®. Variants of these enzymes that can be obtained by point mutations may also be used according to the invention.

The term “cellulase” as used herein refers to an enzyme that catalyzes the hydrolysis of 1,4-β-D-glucoside bonds in cellulose (cellobiose), and/or lichenin and/or β-D-glucans. They are often also able to hydrolyze the 1,4-bonds in β-D-glucans, which also have 1,3-bonds in addition to the 1,4-bonds. Cellulases are able to break down cellulose to β-glucose. Consequently, cellulases act in particular upon cellulose-containing or cellulose derivative-containing functional groups and catalyze their hydrolysis. In a preferred embodiment of the invention, the cellulase is an endoglucanase (EC 3.2.1.4). Synonymous terms can be used for cellulases, in particular endoglucanase, endo-1,4-β-glucanase, carboxymethyl cellulase, endo-1,4-β-D-glucanase, β-1,4-glucanase, β-1,4-endoglucanhydrolase, celludextrinase, or avicelase. The determining factor as to whether an enzyme is a cellulase in the context of the invention is its ability to hydrolyze 1,4-β-D-glucoside bonds in cellulose.

The term “cellulase activity” is defined here as an enzyme that catalyzes the hydrolysis of 1,4-β-D-glucoside bonds into β-1,4-glucan (cellulose). Cellulose activity is measured using a standard method, e.g., as follows: cellulases release glucose from CMC (carboxymethylcellulose). The samples are incubated under defined reaction conditions (100 mM sodium phosphate buffer pH 7.5, 40° C., 15 min) with a substrate (1.25% CMC). The reaction with p-hydroxybenzoic acid hydrazide (PAHBAH) in the presence of bismuth produces a yellow dye that can be determined photometrically at 410 nm. The prerequisite is an alkaline pH during the color reaction. The amount of sugar released corresponding to the coloration is a measure of enzyme activity (Lever, Anal. Biochem., 1972, 47 & 1977, 81).

Suitable cellulases include those of bacterial or fungal origin. Chemically modified or protein-engineered mutants are included. Suitable cellulases are cellulases from the genuses Bacillus, Pseudomonas, Humicola, Fusarium, Thielvia, Acremonium, e.g., the fungal cellulase from Humicola insolens, Mycelophthora thermophila and Fusarium oxysporum, which are disclosed in U.S. Pat. Nos. 4,435,307, 5,648,263, 5,691,178, 5,776,757, and WO 89/09259. Particularly suitable cellulases are the alkaline or neutral cellulases with color care properties. Examples of such cellulases are cellulases which are described in EP 0495257, EP 0531372, WO 96/11262, WO 96/29397 and WO 98/08940. Other examples are cellulase variants as described in WO 94/07998, EP 0531315, EP 3212777, EP 3502243, EP 3653705, EP 3653706, U.S. Pat. Nos. 5,457,046, 5,686,593, 5,763,254, WO 95/24471, WO 98/12307 and WO 99/01544 and WO 2019/122520.

Examples of cellulases with endo-1,4-glucanase activity (EC 3.2.1.4) are described in WO 2002/099091, for example those having a sequence of at least 97% identity to the amino acid sequence of positions 1 to 773 of SEQ ID NO:2 of WO 2002/099091. A further example can comprise a GH44-xyloglucanase, for example a xyloglucanase enzyme having a sequence of at least 60% identity to positions 40 to 559 of SEQ ID NO:2 of WO 2001/062903.

Other examples of cellulases include the GH45 cellulases described in WO 96/29397 and, in particular, variants thereof having substitution, insertion, and/or deletion at one or more of the positions corresponding to the following positions in SEQ ID NO:8 of WO 2002/099091: 2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected from P19A, G20K, Q44K, N48E, Q119H, or Q146R.

Commercially available cellulases include Celluzyme™, Carezyme™, Carezyme Premium™, Celluclean™ (e.g., Celluclean™ 5000L and Celluclean™ 4000T), Celluclean Classic™ Cellusoft™, Endolase®, Renozyme® and Whitezyme™ (Novozymes A/S), Clazinase™ and Puradax HA™ (Genencor International Inc.), KAC-500(B)™ (Kao Corporation), Revitalenz™ 1000, Revitalenz™ 2000 and Revitalenz™ 3000 (DuPont), as well as Ecostone® and Biotouch® (AB Enzymes).

Further enzymes that can be used are, for example, lipases or cutinases, in particular for the triglyceride-cleaving activities thereof, but also so as to create peroxy acids in situ from suitable precursors.

Suitable lipases and cutinases are those of bacterial or fungal origin. Chemically modified mutated enzymes generated by protein engineering are included. Examples are lipase from Thermomyces, e.g., from T. Lanuginosus (formerly called Humicola lanuginosa), as described in EP 0258068 and EP 0305216, cutinase from Humicola, e.g., H. insolens (WO 96/13580), lipase from strains of Pseudomonas (some of these now renamed Burkholderia), e.g., P. alcaligenes or P. pseudoalcaligenes (EP 0218272), P. cephalia (EP 0331376), P. sp. strain SD705 (WO 95/06720 & WO 96/27002), P. wisconsinensis (WO 96/12012), Streptomyces lipases of the GDSL type (WO 2010/065455), cutinase from Magnaporthegrisea (WO 2010/107560), cutinase from Pseudomonas mendocina (U.S. Pat. No. 5,389,536), lipase from Thermobifida fusca (WO 2011/084412), lipase from Geobacillus stearothermophilus (WO 2011/084417), lipase from Bacillus subtilis (WO 2011/084599), and lipase from Streptomycesgriseus (WO 2011/150157) and S. pristinaespiralis (WO 2012/137147).

The lipases that can originally be obtained from Humicola lanuginosa (Thermomyces lanuginosus) or have been developed therefrom, in particular those having one or more of the following amino acid exchanges in positions D96L, T213R and/or N233R, particularly preferably T213R and N233R, proceeding from the mentioned lipase, belong to the preferred lipases. Lipases are marketed, for example, by Novozymes under the trade names Lipolase®, Lipolase® Ultra, LipoPrime®, Lipozyme®, and Lipex®. Another lipase that can be used advantageously is available from Novozymes under the trade name Lipoclean®.

Moreover, the cutinases which have been originally isolated from Fusarium solani pisi and Humicola insolens can also be used, for example. Lipases that are also suitable are available from Amano under the names Lipase CE®, Lipase P®, Lipase B® or Lipase CES®, Lipase AKG®, Bacillus sp. Lipase®, Lipase AP®, Lipase M-AP® and Lipase AML®. From Danisco/DuPont, for example, lipases or cutinases can be used of which the starting enzymes were originally isolated from Pseudomonas mendocina and Fusarium solanii. The preparations M1 Lipase® and Lipomax® marketed by Danisco/Genencor, the enzymes marketed by Meito Sangyo KK, under the names Lipase MY-30®, Lipase OF® and Lipase PL®, and the product Lumafast® from Danisco/DuPont should be mentioned as other important commercial products.

Further examples are lipases, which are also referred to as acyltransferases or perhydrolases, e.g., acyltransferases with homology to Candida antarctica lipase A (WO 2010/111143), acyltransferase from Mycobacterium smegmatis (WO 2005/056782), perhydrolases from the CE 7 family (WO 2009/067279), and variants of M. smegmatis perhydrolase, in particular the S54V variant used in the commercial product Gentle Power Bleach from Huntsman Textile Effects Pte Ltd (WO 2010/100028). Further examples are lipase variants as described in EP 0407225, WO 92/05249, WO 94/01541, WO 94/25578, WO 95/14783, WO 95/30744, WO 95/35381, WO 95/22615, WO 96/00292, WO 97/04079, WO 97/07202, WO 2000/034450, WO 2000/060063, WO 2001/092502, WO 2007/087508, and WO 2009/109500.

Preferred commercial lipase products include Lipolase™, Lipex™, Lipolex™, and Lipoclean™ (Novozymes A/S), Lumafast (Genencor/DuPont), and Lipomax (Gist-Brocades).

In order to increase the bleaching effect, oxidoreductases, such as oxidases, oxygenases, catalases, peroxidases, such as halo, chloro, bromo, lignin, glucose, or manganese peroxidases, dioxygenases or laccases (phenoloxidases, polyphenoloxidases) can be used according to the invention. Advantageously, organic, particularly preferably aromatic compounds that interact with the enzymes are additionally added in order to potentiate the activity of the relevant oxidoreductases (enhancers) or, in the event of greatly differing redox potentials, to ensure the flow of electrons between the oxidizing enzymes and the stains (mediators).

In the cleaning agents described herein, the enzymes to be used can further be formulated together with accompanying substances, for example from fermentation. In liquid formulations, the enzymes are preferably used as liquid enzyme formulation(s).

The enzymes are generally not provided in the form of the pure protein, but rather in the form of stabilized, storable and transportable preparations. These pre-packaged preparations include, for example, the solid preparations obtained through granulation, extrusion, or lyophilization or, in particular in the case of liquid or gel agents, solutions of the enzymes, which are advantageously maximally concentrated, have a low water content, and/or are supplemented with stabilizers or other auxiliaries.

Alternatively, the enzymes can also be encapsulated, for both the solid and the liquid administration form, e.g. by spray-drying or extrusion of the enzyme solution together with a preferably natural polymer or in the form of capsules, e.g. those in which the enzymes are enclosed in a set gel, or in those of the core-shell type, in which an enzyme-containing core is coated with a water-, air-, and/or chemical-impermeable protective layer. Other active ingredients such as stabilizers, emulsifiers, pigments, bleaching agents, or dyes can additionally be applied in overlaid layers. Such capsules are applied using methods that are known per se, for example by shaking or roll granulation or in fluidized bed processes. Advantageously, such granules are low in dust, for example due to the application of polymeric film-formers, and stable in storage due to the coating.

Furthermore, it is possible to formulate two or more enzymes together such that a single granule exhibits a plurality of enzyme activities.

The enzymes can also be introduced into water-soluble films, such as those used in the formulation of washing and cleaning agents in a unit dosage form. Such a film allows the enzymes to be released after contact with water. As used herein, “water-soluble” refers to a film structure that is preferably completely water soluble. Preferably, such a film consists of (completely or partially hydrolyzed) polyvinyl alcohol (PVA).

The invention also relates to a method for cleaning textiles or hard surfaces, which is characterized in that an agent according to the invention is used in at least one method step. In various embodiments, the method described above is characterized in that the protease conjugate is used at a temperature of 0° C. to 100° C., preferably 20° C. to 60° C., more preferably 20° C. to 40° C., and most preferably at 30° C.

This includes both manual and machine methods, with machine methods being preferred because they can be controlled more precisely, for example with regard to the quantities used and contact times. Methods for cleaning textiles are generally characterized by the fact that, in a plurality of method steps, various cleaning-active substances are applied to the material to be cleaned and washed off after the exposure time, or in that the material to be cleaned is otherwise treated with a washing agent or a solution or dilution of this agent.

All aspects, subject matter, and embodiments described for the protease conjugates according to the invention and agents containing them are also applicable to this subject matter of the invention. Therefore, reference is expressly made at this point to the disclosure at the corresponding point with the note that this disclosure also applies to the above methods according to the invention.

### EXAMPLES

### Conjugates According to the Invention Consisting of Protease, Linker and Peptide

With the peptide
(SEQ ID NO: 8)
RALRALQ ALE ALE AL

a. C-terminal peptide: N-protease-rigid linker-peptide-C (the linker is printed in bold and italics for visual differentiation)

AQTVPYGITQIKAPAVHAQGYKGANVKVAVLDTGIHAAHPDLNVAGGAS
FVPSEPNATQDFQSHGTHVAGTIAALDNTIGVLGVAPSAALYAVKVLDR
NGDGQYSWIISGIEWAVANNMDVINMSLGGPDGSAALKNAVDTANKRGV
VVVAAAGNSGSTGSTSTVGYPAKYDSTIAVANVNSSNVRNSTSSAGPEL
DVSAPGTSILSTVPSSGYTSMTGTSMAAPHVAGAAALILSKNPNLSNSQ
VRQRLETTATPLGNSFYYGKGLINAEAASNEAAAKEAAAKEAAAKRALR
ALQALEALEAL

b. C-terminal peptide: N-protease-flexible linker-peptide-C (the linker is printed in bold and italics for visual differentiation

AQTVPYGITQIKAPAVHAQGYKGANVKVAVLDTGIHAAHPDLNVAGGAS
FVPSEPNATQDFQSHGTHVAGTIAALDNTIGVLGVAPSAALYAVKVLDR
NGDGQYSWIISGIEWAVANNMDVINMSLGGPDGSAALKNAVDTANKRGV
VVVAAAGNSGSTGSTSTVGYPAKYDSTIAVANVNSSNVRNSTSSAGPEL
DVSAPGTSILSTVPSSGYTSMTGTSMAAPHVAGAAALILSKNPNLSNSQ
VRQRLETTATPLGNSFYYGKGLINAEAASNGGGGSGGGGSGGGGSRALR
ALQALEALEAL

c. N-terminal peptide: N-peptide-rigid linker-protease-C (the linker is printed in bold and italics for visual differentiation)

RALRALQALEALEALEAAAKEAAAKEAAAKAQTVPYGITQIKAPAVHAQ
GYKGANVKVAVLDTGIHAAHPDLNVAGGASFVPSEPNATQDFQSHGTHV
AGTIAALDNTIGVLGVAPSAALYAVKVLDRNGDGQYSWIISGIEWAVAN
NMDVINMSLGGPDGSAALKNAVDTANKRGVVVVAAAGNSGSTGSTSTVG
YPAKYDSTIAVANVNSSNVRNSTSSAGPELDVSAPGTSILSTVPSSGYT
SMTGTSMAAPHVAGAAALILSKNPNLSNSQVRQRLETTATPLGNSFYYG
KGLINAEAASN

d. N-terminal peptide: N-peptide-flexible linker-protease-C (the linker is printed in bold and italics for visual differentiation)

RALRALQALEALEALGGGGSGGGGSGGGGSAQTVPYGITQIKAPAVHA
QGYKGANVKVAVLDTGIHAAHPDLNVAGGASFVPSEPNATQDFQSHGT
HVAGTIAALDNTIGVLGVAPSAALYAVKVLDRNGDGQYSWIISGIEWA
VANNMDVINMSLGGPDGSAALKNAVDTANKRGVVVVAAAGNSGSTGST
STVGYPAKYDSTIAVANVNSSNVRNSTSSAGPELDVSAPGTSILSTVP
SSGYTSMTGTSMAAPHVAGAAALILSKNPNLSNSQVRQRLETTATPLG
NSFYYGKGLINAEAASN
With the peptide
(SEQ ID NO: 24)
WRHPRLRCGNLL

a. C-terminal peptide: N-protease-rigid linker-peptide-C (the linker is printed in bold and italics for visual differentiation)

AQTVPYGITQIKAPAVHAQGYKGANVKVAVLDTGIHAAHPDLNVAGGA
SFVPSEPNATQDFQSHGTHVAGTIAALDNTIGVLGVAPSAALYAVKVL
DRNGDGQYSWIISGIEWAVANNMDVINMSLGGPDGSAALKNAVDTANK
RGVVVVAAAGNSGSTGSTSTVGYPAKYDSTIAVANVNSSNVRNSTSSA
GPELDVSAPGTSILSTVPSSGYTSMTGTSMAAPHVAGAAALILSKNPN
LSNSQVRQRLETTATPLGNSFYYGKGLINAEAASNEAAAKEAAAKEAA
AK WRHPRLRCGNLL

b. C-terminal peptide: N-protease-flexible linker-peptide-C (the linker is

AQTVPYGITQIKAPAVHAQGYKGANVKVAVLDTGIHAAHPDLNVAGGA
SFVPSEPNATQDFQSHGTHVAGTIAALDNTIGVLGVAPSAALYAVKVL
DRNGDGQYSWIISGIEWAVANNMDVINMSLGGPDGSAALKNAVDTANK
RGVVVVAAAGNSGSTGSTSTVGYPAKYDSTIAVANVNSSNVRNSTSSA
GPELDVSAPGTSILSTVPSSGYTSMTGTSMAAPHVAGAAALILSKNPN
LSNSQVRQRLETTATPLGNSFYYGKGLINAEAASNGGGGSGGGGSGGG
GS WRHPRLRCGNLL

c. N-terminal peptide: N-peptide-rigid linker-protease-C (the linker is printed in bold and italics for visual differentiation)

WRHPRLRCGNLLEAAAKEAAAKEAAAKAQTVPYGITQIKAPAVHAQGY
KGANVKVAVLDTGIHAAHPDLNVAGGASFVPSEPNATQDFQSHGTHVA
GTIAALDNTIGVLGVAPSAALYAVKVLDRNGDGQYSWIISGIEWAVAN
NMDVINMSLGGPDGSAALKNAVDTANKRGVVVVAAAGNSGSTGSTSTV
GYPAKYDSTIAVANVNSSNVRNSTSSAGPELDVSAPGTSILSTVPSSG
YTSMTGTSMAAPHVAGAAALILSKNPNLSNSQVRQRLETTATPLGNSF
YYGKGLINAEAASN

d. N-terminal peptide: N-peptide-flexible linker-protease-C (the linker is printed in bold and italics for visual differentiation)

WRHPRLRCGNLLGGGGSGGGGSGGGGSAQTVPYGITQIKAPAVHAQGY
KGANVKVAVLDTGIHAAHPDLNVAGGASFVPSEPNATQDFQSHGTHVA
GTIAALDNTIGVLGVAPSAALYAVKVLDRNGDGQYSWIISGIEWAVAN
NMDVINMSLGGPDGSAALKNAVDTANKRGVVVVAAAGNSGSTGSTSTV
GYPAKYDSTIAVANVNSSNVRNSTSSAGPELDVSAPGTSILSTVPSSG
YTSMTGTSMAAPHVAGAAALILSKNPNLSNSQVRQRLETTATPLGNSF
YYGKGLINAEAASN

Compared to the wild-type protease (SEQ ID NO:3), variant 1 has the following mutations:

| | HP388 | | | | | | | | | |
|---|---|---|---|---|---|---|---|---|---|---|
| | P9 | N130 | Q271 | N144 | N252 | Y217 | T133 | S224 | S189 | S89 |
| HP545 | T | D | E | K | T | M | A | A | T | A |

### Washing Agent Matrix Used

The following table shows the washing agent matrix (contains enzymes but no protease) used for the washing test:

| | Amount of active | Amount of active |
|---|---|---|
| | substance in raw | substance in the |
| Chemical name | material [wt. %] | formulation [wt. %] |
| Demineralized water | 100 | Remainder |
| Citric acid | 100 | 1-5 |
| Defoamer | 100 | <1 |
| Fatty alcohol ether sulfate | 70 | 3-8 |
| Fatty alcohol, ethoxylated | 100 | 2-11 |
| Alkylbenzene sulfonate | 96 | 3-20 |
| Fatty acid | 30 | 0.3-4 |
| NaOH | 50 | 0.5-2 |
| Glycerol | 99.5 | 1-3 |
| 1,2-propanediol | 100 | 8-12 |
| HEDP | 60 | 0.5-2 |
| Soil repellent polymer | 30 | 0.1-1 |
| Stabilizer | 100 | 0.1-0.5 |
| Enzymes (except protease), | t.q. | Low |
| perfume, DTI, optical | | |
| brightener | | |
| pH 8.2-8.4; dosage | | |
| 3.17 g/L | | |

### Protease Activity Assays

Protease activity is determined in a discontinuous assay using casein as substrate. The final concentration of the substrate solution was 12 mg/ml casein (prepared according to Hammarsten; Merck, Darmstadt, #2242) and 30 mM Tris in synthetic tap water. Synthetic tap water is a solution of 0.029% (w/v) CaCl₂·2H₂O, 0.014% (w/v) MgCl₂·6H₂O and 0.021% (w/v) NaHCO₃ with 15′ dH (German hardness). The substrate solution is heated to 70° C. and the pH is adjusted to 8.5 at 50° C. by using 0.1 N NaOH. The protease solution is prepared by adding 2% (w/v) water-free pentasodium tripolyphosphate to synthetic tap water and adjusting to pH 8.5 with hydrochloric acid. 200 μl of the enzyme solution is added to 600 μl of the casein solution. The mixture is incubated at 50° C. for 15 minutes. The reaction is terminated by the addition of 600 μl of 0.44 M trichloroacetic acid (TCA), 0.22 M sodium acetate in 3% (w/v). After a cooling step of 15 minutes on ice, the TCA insoluble protein is removed by centrifugation. 900 μl of the remaining solution is mixed with 300 μl of 2 N NaOH and the absorption of this mixture containing TCA-soluble proteins is measured at 290 nm. Control values are generated by adding 600 μl TCA solution to 600 μl casein solution, followed by addition of 200 μl enzyme solution. A protease solution which causes an absorption change of 0.500 OD at 290 nm under these conditions has, according to the present designation, an activity of 10 HPE per ml.

### Mini Washing Test and Results

Mini wash test was carried out with Bacillus subtilis culture supernatants containing the protease conjugates. The supernatants are washed with the same activity as the benchmark (here variant 1): 6 HPE/ml (=1.15 μg/ml active protein in the wash liquor).

### Conditions:

40° C., 16° dH water, 1 h

### Stains:

CS38: egg yolk pigment
ION: whole egg, soot
C05: blood, milk, ink
CS32: sebum
PC10: milk, oil
H-MR-B: milk, soot

Punched-out pieces of fabric (diameter=10 mm) were placed in a microtiter plate, washing liquor was preheated to 40° C., with a final concentration of 3.17 g/L, the liquor and enzyme (1.15 μg/mL) were applied to the stain and incubated for 1 h at 40° C. and 600 rpm, then the stain was rinsed repeatedly with clear water and left to dry and the brightness was determined using a color measuring device. The lighter the fabric, the better the cleaning performance. The Y value=brightness is measured here, the higher the brighter. All measured Y values were corrected by the performance of the wash liquor alone (without protease) (Y-variant−Y-blank=ΔY-variant). For each stain, a AY-variant was determined for each protease and all AY values of the 6 stains per variant were summed to determine ΣΔY variant.

### Results Wash Performance at 40° C.

| | Peptide | | | | | | | | | | | | | |
|---|---|---|---|---|---|---|---|---|---|---|---|---|---|---|
| | x | SEQ ID NO: 8 | | | | SEQ ID NO: 24 | | | | SEQ ID NO: 28 | | | | x |
| | Term | | | | | | | | | | | | | |
| | N/C | C | C | N | N | C | C | N | N | C | C | N | N | x |
| | Linker | | | | | | | | | | | | | |
| | x | A** | B*** | B | A | A | B | B | A | A | B | B | A | x |
| | Prot.* | | | | | | | | | | | | | |
| | ✓ | ✓ | ✓ | ✓ | ✓ | ✓ | ✓ | ✓ | ✓ | ✓ | ✓ | ✓ | ✓ | x |
| CS38 | 12.08 | 12.79 | 12.17 | 12.87 | 12.84 | 12.61 | 11.84 | 13.41 | 12.93 | 13.46 | 13.30 | 14.93 | 13.82 | 1.44 |
| 10 N | 8.51 | 8.45 | 8.01 | 8.28 | 8.33 | 9.40 | 8.51 | 8.76 | 7.39 | 7.98 | 9.47 | 11.87 | 9.12 | 2.76 |
| C05 | 6.64 | 5.72 | 6.20 | 6.08 | 6.87 | 6.09 | 6.22 | 6.88 | 6.28 | 5.79 | 6.06 | 7.78 | 7.85 | 0.63 |
| CS32 | 0.27 | 0.57 | −0.33 | 0.64 | −0.30 | 0.23 | −0.54 | −0.51 | 0.48 | −0.79 | −0.64 | 2.53 | 0.48 | −0.60 |
| PC10 | 12.73 | 12.20 | 16.20 | 16.67 | 19.25 | 11.02 | 16.79 | 15.39 | 10.39 | 14.33 | 15.89 | 8.77 | 18.56 | −0.89 |
| H-MR-B | 10.10 | 12.07 | 11.40 | 13.55 | 11.09 | 11.05 | 12.35 | 9.31 | 10.39 | 10.67 | 12.58 | 11.58 | 13.09 | −0.76 |
| ΣΔY | 49.78 | 51.81 | 53.65 | 58.09 | 58.09 | 50.40 | 55.17 | 53.23 | 47.86 | 51.44 | 56.66 | 57.46 | 62.91 | 2.57 |

*Variant 1;
**(EAAAK)₃;
***(GGGGS)₃

In general, the fusion constructs show a significantly increased wash performance compared to the starting protease, regardless of the peptide or linker. The protease activity is improved in particular in combination with the peptides SEQ ID NO. 24 and SEQ ID NO. 28 when located at the N-terminus of the protease, independently of the linkers.

1. A protease conjugate consisting of
A) a protease of the subtilisin type from Bacillus pumilus, wherein the protease has proteolytic activity;
B) at least one heterologous peptide covalently linked to the protease; and optionally
C) at least one peptide linker;
wherein the heterologous peptide is a peptide comprising an amino acid sequence of 4 to 50 amino acids, wherein
(a) the amino acid sequence in N- to C-terminal orientation has the following sequence
(C)mX1X2X3(X4)nX5(C)o,
where X1 is a positively charged amino acid, X2 and X3 are uncharged amino acids, each X4 is, independently of one another, any amino acid; X5 is any positively charged or uncharged amino acid, m and o are 0 or 1, where m+o 0 or 1; and n is an integer from 0 to 46;
or
(b) the amino acid sequence has at least 80% sequence identity to one of the amino acid sequences specified in SEQ ID NOs: 19-20 or 21-25.

2. The protease conjugate according to claim 1, wherein
(i) the peptide has a total charge of 0 to +4; and/or
(ii) the N-terminus comprising the first 3-4 amino acids has a net positive charge; and/or
(iii) the C-terminus comprising the last 3-6 amino acids has a net negative or neutral charge; and/or
(iv) the peptide contains no P.

3. The protease conjugate according to claim 1, where the peptide has the amino acid sequence according to one of SEQ ID NOs: 4-30, as well as variants thereof, which have at least 80% sequence identity to the specified sequence, with the RAL motif, and also the EAL and/or QAL motif, if present, being invariable.

4. The protease conjugate according to claim 1, where the peptide has the amino acid sequence according to SEQ ID NO:24 or according to SEQ ID NO:28, as well as variants thereof, which have at least 80%, sequence identity to the specified sequence, with the RAL motif, and also the EAL and/or QAL motif, if present, being invariable.

5. The protease conjugate according to claim 1, wherein the protease is selected from:
a) a protease that has proteolytic activity and comprises an amino acid sequence which is at least 70% identical to the amino acid sequence given in SEQ ID NO:1 over its entire length and, respectively based on the numbering according to SEQ ID NO:2 has, (i) the amino acid substitution R101E, and (ii), at least one amino acid substitution selected from the group consisting of: S3T, V4I, R45E, R45D, R45Q, P55N, T58W, T58Y, T58L, Q59D, Q59M, Q59N, Q59T, G61D, G61R, S87E, G97S, A98D, A98E, A98R, S106A, S106W, N117E, H120V, H120D, H120K, H120N, S124M, P129D, E136Q, Q137H, S143W, S156D, S161T, S163A, S163G, Y171L, A172S, N185Q, V199M, V205I, Y209W, M222Q, N238H, V244T, N261T and L262N, L262Q, L262D, L262E; wherein the amino acid substitution combination of group (ii) is selected from the group consisting of N238E-L262E, S156D-L262E, S3T-V41-V2051, S3T-V41-A228V, G195E-V199M, H120D-S163G-N261D, N76D-A228V-N261D, S3T-N76D-S156D-Y209W, Q137H-S141H-R145H-N238E-L262E, Q137H-S141H-R145H-S156D-L262E, N76D-Q137H-S141H-R145H-A228V-N261D, N76D-Q137H-S141H-R145H-S163G-N238E, H120D-Q137H-S141H-R145H-S163G-N261D, S3T-N76D-Q137H-S141H-R145H-S156D-Y209W; or
b) a protease that has proteolytic activity and comprises an amino acid sequence which is at least 70% identical to the amino acid sequence given in SEQ ID NO:3 over its entire length and at least one amino acid substitution selected from the group consisting of P9T, N130D, N130V, T133A, N144K, Y217M, N252T and Q271E, and (ii), at least one amino acid substitution selected from the group consisting of Y6F, Y6W, F61G, Q62N, S63Q, S89A, S89G, N99H, D101S, D101E, D101A, G131H, G131Y, G131F, S156R, G166A, G166M, G166L, G166I, K170R, K170G, N187D, N188G, S189T, S189L, S189I, S189R, S211N, S211Q, S224A, S224G, wherein the protease has an amino acid substitution combination selected from the group consisting of: P9T-N130D-T133A-N144K-G166M-S189T-Y217M-N252T-Q271E, P9T-N130D-T133A-N144K-G166M-S189T-Y217M-S224A-N252T-Q271E, P9T-N130D-T133A-N144K-G166M-S211N-Y217M-N252T-Q271E, P9T-S89A-N130D-G131H-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-N187D-Y217M-S224A-N252T-Q271E, P9T-S89A-N130D-T133A-N144K-S189R-Y217M-S224A-N252T-Q271E, P9T-S89A-N130D-T133A-N144K-N187D-S189R-Y217M-S224A-N252T-Q271E, P9T-S89A-D101S-N130D-T133A-N144K-S189T-Y217M-S224A-N252T-Q271E, P9T-S89A-D101E-N130D-T133A-N144K-S189T-Y217M-S224A-N252T-Q271E, P9T-S89A-D101A-N130D-T133A-N144K-S189T-Y217M-S224A-N252T-Q271E, P9T-S89A-N99H-N130D-T133A-N144K-K170R-S189T-Y217M-S224A-N252T-Q271E, P9T-S89A-N130D-T133A-N144K-S156R-S189T-Y217M-S224A-N252T-Q271E, P9T-S63Q-S89A-N130D-T133A-N144K-G166A-S189T-Y217M-S224A-N252T-Q271E, P9T-F61G-Q62-N-S89A-N130D-T133A-N144K-N188G-S189T-Y217M-S224A-N252T-Q271E, Y6W-P9T-N130D-T133A-N144K-S189T-Y217M-S224A-N252T-Q271E, Y6W-P9T-S89A-N130D-T133A-N144K-S189T-Y217M-S224A-N252T-Q271E, Y6W-P9T-F61G-Q62N-S89A-N130D-T133A-N144K-S189T-Y217M-S224A-N252T-Q271E, Y6W-P9T-S89A-N130D-T133A-N144K-N188G-S189T-Y217M-S224A-N252T-Q271E; or
c) a protease that has proteolytic activity and comprises an amino acid sequence which is at least 70% to the amino acid sequence given in SEQ ID NO:1 over its entire length and, respectively based on the numbering according to SEQ ID NO:1 (i) the amino acid substitutions S3T, V41, R99E and V199I, and (ii), at least one amino acid substitution selected from the group consisting of N74D, N74E, N74Q, A136Q, R143L, R143W, R143Y, S154D, S154Q, S160G, Y161T, A163G, V171L, A181D, F183R, Q185R, Q200A, Q200L, Q200S, Q200T, Y203K, Y203V, Y203W, A209K, A209W, N212S, N212T and L256D, L256E and L256Q, wherein the protease has an amino acid substitution combination selected from the group consisting of S3T-V4I-R99E-V199I-Q200L-Y203W, S3T-V4I-R99E-V199I-N212S, S3T-V4I-R99E-V199I-N74D, S3T-V4I-R99E-V199I-S154D-L256E, S3T-V4I-R99E-V199I-Q200L-Y203W-S154D-L256E, S3T-V41-R99E-V199I-N74D-Q200L-Y203W, S3T-V4I-R99E-V199I-N74D-S154D-Q200L-Y203W-L256E, S3T-V4I-R99E-V199I-N74D-N212S, S3T-V4I-R99E-V199I-N74D-S154D-Y203W-L256E, S3T-V4I-R99E-V199I-N74D-Y203W, S3T-V4I-R99E-V199I-N74D-S154D-Q200L-L256E, S3T-V4I-R99E-V199I-N74D-Q200L, S3T-V4I-R99E-V199I-S154D-Q200L-Y203W, S3T-V41-R99E-V199I-Q200L-Y203W-L256E, S3T-V4I-R99E-V199I-A136Q-R143W-Y161T-Q200L, S3T-V4I-R99E-V199I-N74D-R143Y-A209W-N212S-L256E, S3T-V41-R99E-V199I-N74D-S154D-Y203W-L256E; S3T-V4I-R99E-V199I-Q200L-Y203W-A209K-S154D-L256E, S3T-V4I-R99E-V199I-S154D-S160G-Q185R-Q200L-Y203W-L256E, S3T-V41-R99E-V199I-S154D-A181D-F183R-Q200L-Y203W-L256E and S3T-V41-R99E-V199I-A136Q-S154D-V171L-Q200L.

6. The protease conjugate according to claim 1, wherein the protease is selected from a protease that has proteolytic activity and comprises an amino acid sequence which is at least 70% identical to the amino acid sequence given in SEQ ID NO:3 over its entire length and, respectively based on the numbering according to SEQ ID NO:2 has, (i) at least one amino acid substitution selected from the group consisting of P9T, N130D, N130V, T133A, N144K, Y217M, N252T and Q271E, and (ii), at least one amino acid substitution selected from the group consisting of Y6F, Y6W, F61G, Q62N, S63Q, S89A, S89G, N99H, D101S, D101E, D101A, G131H, G131Y, G131F, S156R, G166A, G166M, G166L, G1661, K170R, K170G, N187D, N188G, S189T, S189L, S1891, S189R, S211N, S211Q, S224A, and S224G; and, wherein the protease has an amino acid substitution combination selected from the group consisting of: P9T-N130D-T133A-N144K-G166M-S189T-Y217M-N252T-Q271E, P9T-N130D-T133A-N144K-G166M-S189T-Y217M-S224A-N252T-Q271E, P9T-N130D-T133A-N144K-G166M-S211N-Y217M-N252T-Q271E, P9T-S89A-N130D-G131H-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-N187D-Y217M-S224A-N252T-Q271E, P9T-S89A-N130D-T133A-N144K-S189R-Y217M-S224A-N252T-Q271E, P9T-S89A-N130D-T133A-N144K-N187D-S189R-Y217M-S224A-N252T-Q271E, P9T-S89A-D101S-N130D-T133A-N144K-S189T-Y217M-S224A-N252T-Q271E, P9T-S89A-D101E-N130D-T133A-N144K-S189T-Y217M-S224A-N252T-Q271E, P9T-S89A-D101A-N130D-T133A-N144K-S189T-Y217M-S224A-N252T-Q271E, P9T-S89A-N99H-N130D-T133A-N144K-K170R-S189T-Y217M-S224A-N252T-Q271E, P9T-S89A-N130D-T133A-N144K-S156R-S189T-Y217M-S224A-N252T-Q271E, P9T-S63Q-S89A-N130D-T133A-N144K-G166A-S189T-Y217M-S224A-N252T-Q271E, P9T-F61G-Q62-N-S89A-N130D-T133A-N144K-N188G-S189T-Y217M-S224A-N252T-Q271E, Y6W-P9T-N130D-T133A-N144K-S189T-Y217M-S224A-N252T-Q271E, Y6W-P9T-S89A-N130D-T133A-N144K-S189T-Y217M-S224A-N252T-Q271E, Y6W-P9T-F61G-Q62N-S89A-N130D-T133A-N144K-S189T-Y217M-S224A-N252T-Q271E, and Y6W-P9T-S89A-N130D-T133A-N144K-N188G-S189T-Y217M-S224A-N252T-Q271E.

7. The protease conjugate according to claim 1, wherein the heterologous peptide sequence is linked to the protease via a peptide linker, selected from flexible linkers and rigid linkers, wherein the protease conjugate has the following structure in N- to C-terminal orientation:
(i) peptide linker protease; or
(ii) protease linker peptide.

8. The protease conjugate according to claim 7, wherein the linker is selected from the group consisting of (GGGGS)n with n=1, 2, 3, or 4; (G)6; (G)8; (EAAAK)n with n=1, 2, or 3; A(EAAAK)4ALEA(EAAAK)4A; PAPAP; AEAAAKEAAAKA; and (AP)n with n=10-34.

9. A nucleic acid that codes for a protease conjugate according to claim 1.

10. A non-human host cell containing a a protease conjugate according to claim 1.

11. A method for producing a protease conjugate, comprising
a) cultivating a host cell according to claim 10; and
b) isolating the protease conjugate from the culture medium or from the host cell.

12. A washing or cleaning agent, characterized in that it contains at least one protease conjugate according to claim 1.

13. A method for cleaning textiles or hard surfaces, characterized in that an agent according to claim 12 is used in at least one method step.

14. The use of a protease conjugate according to claim 1 in a washing or cleaning agent for removing peptide- or protein-containing stains.

15. A non-human host cell containing a nucleic acid according to claim 9.


SEQUENCE LISTING (57 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus amyloliquefaciens [sequence omitted]

SEQ ID NO: 3 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSSGYTSYTGTSMASPHVAGAAALILSKNPNLSNSQVRQRLENTATPLGNSFYYGKGLINAQAASN

SEQ ID NO: 4 - protein, 15 aa, synthetic construct
  source 1..15: note = Peptide RAL-M
RALQALRALQALEAL

SEQ ID NO: 5 - protein, 15 aa, synthetic construct
  source 1..15: note = Peptide RAL2E
RALRALRALEALEAL

SEQ ID NO: 6 - protein, 15 aa, synthetic construct
  source 1..15: note = Peptide RAL2Q
RALRALRALQALQAL

SEQ ID NO: 7 - protein, 15 aa, synthetic construct
  source 1..15: note = Peptide RALQE
RALRALRALQALEAL

SEQ ID NO: 8 - protein, 15 aa, synthetic construct
  source 1..15: note = Peptide RALQ2E
RALRALQALEALEAL

SEQ ID NO: 9 - protein, 17 aa, synthetic construct
  source 1..17: note = Peptide RAL2FQ
RALFEALQALFRALEAL

SEQ ID NO: 10 - protein, 14 aa, synthetic construct
  source 1..14: note = Peptide RALEQE
RALRALEALQALEA

SEQ ID NO: 11 - protein, 15 aa, synthetic construct
  source 1..15: note = Peptide RAL2FR
RALFEALFRALEALR

SEQ ID NO: 12 - protein, 14 aa, synthetic construct
  source 1..14: note = Peptide RAL2F
RALFEALFRALEAL

SEQ ID NO: 13 - protein, 13 aa, synthetic construct
  source 1..13: note = Peptide RALF
RALEALFRALEAL

SEQ ID NO: 14 - protein, 13 aa, synthetic construct
  source 1..13: note = Peptide RALF_sym
RALRALFEALEAL

SEQ ID NO: 15 - protein, 16 aa, synthetic construct
  source 1..16: note = Peptide RALF_Q
RALEALFRALQALEAL

SEQ ID NO: 16 - protein, 16 aa, synthetic construct
  source 1..16: note = Peptide RALFQ_W
RALEALWRALQALEAL

SEQ ID NO: 17 - protein, 13 aa, synthetic construct
  source 1..13: note = Peptide RALF_W
RALEALWRALEAL

SEQ ID NO: 18 - protein, 16 aa, synthetic construct
  source 1..16: note = Peptide RALA4Q
RALARALARALAQALA

SEQ ID NO: 19 - protein, 15 aa, synthetic construct
  source 1..15: note = Peptide P8_S2
RSIVTFSLRQNAQLA

SEQ ID NO: 20 - protein, 15 aa, synthetic construct
  source 1..15: note = PeptideP8_S8
RSIVTFSLRQNSEQA

SEQ ID NO: 21 - protein, 12 aa, synthetic construct
  source 1..12: note = Peptide BA1-A06
GLHTSATNLYLH

SEQ ID NO: 22 - protein, 12 aa, synthetic construct
  source 1..12: note = Peptide D2
QHSIRLLTIKKP

SEQ ID NO: 23 - protein, 12 aa, synthetic construct
  source 1..12: note = Peptide D4
QQSIRIMTIKHP

SEQ ID NO: 24 - protein, 12 aa, synthetic construct
  source 1..12: note = Peptide P3
WRHPRLRCGNLL

SEQ ID NO: 25 - protein, 12 aa, synthetic construct
  source 1..12: note = Peptide P14
QKSRNRMTRTHP

SEQ ID NO: 26 - protein, 12 aa, synthetic construct
  source 1..12: note = Peptide P6
SRARLFVVTYHK

SEQ ID NO: 27 - protein, 12 aa, synthetic construct
  source 1..12: note = Peptide P7
HMISTMNAASRR

SEQ ID NO: 28 - protein, 12 aa, synthetic construct
  source 1..12: note = Peptide P8
RSIVTFSLRQNR

SEQ ID NO: 29 - protein, 12 aa, synthetic construct
  source 1..12: note = Peptide P13
RNTIRIRTIKHP

SEQ ID NO: 30 - protein, 12 aa, synthetic construct
  source 1..12: note = Peptide P15
RHSSTLRYRPLP

SEQ ID NO: 31 - protein, 16 aa, synthetic construct
  source 1..16: note = Peptide RAL-M-C
RALQALRALQALEALC

SEQ ID NO: 32 - protein, 16 aa, synthetic construct
  source 1..16: note = Peptide RAL2E-C
RALRALRALEALEALC

SEQ ID NO: 33 - protein, 16 aa, synthetic construct
  source 1..16: note = Peptide RAL2Q-C
RALRALRALQALQALC

SEQ ID NO: 34 - protein, 16 aa, synthetic construct
  source 1..16: note = Peptide RALQE-C
RALRALRALQALEALC

SEQ ID NO: 35 - protein, 16 aa, synthetic construct
  source 1..16: note = Peptide RALQ2E-C
RALRALQALEALEALC

SEQ ID NO: 36 - protein, 18 aa, synthetic construct
  source 1..18: note = Peptide RAL2FQ-C
RALFEALQALFRALEALC

SEQ ID NO: 37 - protein, 15 aa, synthetic construct
  source 1..15: note = Peptide RALEQE-C
RALRALEALQALEAC

SEQ ID NO: 38 - protein, 16 aa, synthetic construct
  source 1..16: note = Peptide RAL2FR-C
RALFEALFRALEALRC

SEQ ID NO: 39 - protein, 15 aa, synthetic construct
  source 1..15: note = Peptide RAL2F-C
RALFEALFRALEALC

SEQ ID NO: 40 - protein, 14 aa, synthetic construct
  source 1..14: note = Peptide RALF-C
RALEALFRALEALC

SEQ ID NO: 41 - protein, 14 aa, synthetic construct
  source 1..14: note = Peptide RALF_sym
RALRALFEALEALC

SEQ ID NO: 42 - protein, 17 aa, synthetic construct
  source 1..17: note = Peptide RALF_Q-C
RALEALFRALQALEALC

SEQ ID NO: 43 - protein, 17 aa, synthetic construct
  source 1..17: note = Peptide RALFQ_W-C
RALEALWRALQALEALC

SEQ ID NO: 44 - protein, 14 aa, synthetic construct
  source 1..14: note = Peptide RALF_W-C
RALEALWRALEALC

SEQ ID NO: 45 - protein, 17 aa, synthetic construct
  source 1..17: note = Peptide RALA4Q-C
RALARALARALAQALAC

SEQ ID NO: 46 - protein, 16 aa, synthetic construct
  source 1..16: note = Peptide P8_S2-C
RSIVTFSLRQNAQLAC

SEQ ID NO: 47 - protein, 16 aa, synthetic construct
  source 1..16: note = Peptide P8_S8-C
RSIVTFSLRQNSEQAC

SEQ ID NO: 48 - protein, 13 aa, synthetic construct
  source 1..13: note = Peptide BA1-A06-C
GLHTSATNLYLHC

SEQ ID NO: 49 - protein, 13 aa, synthetic construct
  source 1..13: note = Peptide D2-C
QHSIRLLTIKKPC

SEQ ID NO: 50 - protein, 13 aa, synthetic construct
  source 1..13: note = Peptide D4-C
QQSIRIMTIKHPC

SEQ ID NO: 51 - protein, 13 aa, synthetic construct
  source 1..13: note = Peptide P3-C
WRHPRLRCGNLLC

SEQ ID NO: 52 - protein, 13 aa, synthetic construct
  source 1..13: note = Peptide P14-C
QKSRNRMTRTHPC

SEQ ID NO: 53 - protein, 13 aa, synthetic construct
  source 1..13: note = Peptide P6-C
SRARLFVVTYHKC

SEQ ID NO: 54 - protein, 13 aa, synthetic construct
  source 1..13: note = Peptide P7-C
HMISTMNAASRRC

SEQ ID NO: 55 - protein, 13 aa, synthetic construct
  source 1..13: note = Peptide P8-C
RSIVTFSLRQNRC

SEQ ID NO: 56 - protein, 13 aa, synthetic construct
  source 1..13: note = Peptide P13-C
RNTIRIRTIKHPC

SEQ ID NO: 57 - protein, 13 aa, synthetic construct
  source 1..13: note = Peptide P15-C
RHSSTLRYRPLPC